(19) United States
(12) Patent Application Publication
(10) Pub. No.: US 2024/0016641 A1
(43) Pub. Date: Jan. 18, 2024

(54) Dynamic Leash for Dynamic Gastric Bypass Device

(71) Applicants: Boston Scientific Scimed, Inc., Maple Grove, MN (US); Mayo Foundation for Medical Education and Research, Rochester, MN (US)

(72) Inventors: Roger W. McGowan, Otsego, MN (US); Jessica Brand, MAPLE GROVE, MN (US); Daniel Lee Krautkremer, Plymouth, MN (US); Alexander James Wiedmann, Columbia Heights, MN (US); Barham K. Abu Dayyeh, Rochester, MN (US); Benjamin Moses Cooke, Ithica, NY (US); Burton Hedstrom, Minnetonka, MN (US); Leo Joseph Steiner, Boston, MA (US)

(73) Assignees: Boston Scientific Scimed, Inc., Maple Grove, MN (US); Mayo Foundation for Medical Education and Research, Rochester, MN (US)

(21) Appl. No.: 18/354,304

(22) Filed: Jul. 18, 2023

Related U.S. Application Data
(60) Provisional application No. 63/390,183, filed on Jul. 18, 2022.

(51) Int. Cl. A61F 5/00; A61B 18/14

(52) U.S. Cl. CPC A61F 5/0079; A61B 18/14; A61F 5/0089; A61B 2018/00595

(57) ABSTRACT

A gastric bypass device includes an occlusion device that is adapted to be deployed relative to a patient's pyloric sphincter, an anastomosis anchor that is adapted to be deployed relative to an anastomosis between the patient's stomach and their small intestine, and a tether that extends through the patient's small intestine between the occlusion device and the anastomosis anchor. A dynamic leash may be secured relative to the occlusion device and may work in conjunction with the tether to help hold the occlusion device in place.

### CROSS-REFERENCE TO RELATED APPLICATIONS

This application claims the benefit of priority under 35 U.S.C. § 119 of U.S. Provisional Application No. 63/390,183, filed Jul. 18, 2022, the entire disclosure of which is hereby incorporated by reference.

### TECHNICAL FIELD

The present disclosure pertains to medical devices such as gastric bypass devices. More particularly, the present disclosure pertains to medical devices such as gastric bypass devices including dynamic leashes.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

The disclosure pertains to medical devices such as gastric bypass devices and more particularly to medical devices such as gastric bypass devices including dynamic leashes.

An example may be found in an implantable medical device system. The implantable medical device system includes an occlusion device adapted to be secured in place relative to a patient's pylorus and an anastomosis anchor adapted to be secured in place relative to an anastomosis formed between the patient's stomach wall and the patient's small intestine. A tether is adapted to extend through the patient's duodenum, the tether secured at a first end to the occlusion device and at a second end to the anastomosis anchor, the tether further adapted to provide a tensile force to the occlusion device. A dynamic leash is adapted to extend through the patient's stomach, the dynamic leash further adapted to provide a tensile force to the occlusion device that is in opposition to that provided by the tether.

Alternatively or additionally, the tether may include a first spring with a first spring constant and the dynamic leash may include a second spring with a second spring constant.

Alternatively or additionally, the second spring constant may be different from the first spring constant.

Alternatively or additionally, the first spring may be adapted to resist gastrointestinal system movements that could otherwise cause the occlusion device to move into the patient's stomach.

Alternatively or additionally, the second spring may be adapted to resist gastrointestinal system movements that could otherwise cause the occlusion device to move into the patient's duodenum.

Alternatively or additionally, the first spring may be adapted to work together with the second spring to keep the occlusion device in place relative to the patient's pylorus.

Alternatively or additionally, the dynamic leash may be adapted to be secured at one end to the occlusion device and at an opposing end to the anastomosis anchor.

Alternatively or additionally, the occlusion device may include a supporting frame and an impermeable membrane spanning the supporting frame.

Alternatively or additionally, the dynamic leash may be adapted to be secured at one end to the supporting frame of the occlusion device.

Alternatively or additionally, the dynamic leash may be adapted to be secured at one end to the impermeable membrane of the occlusion device.

Alternatively or additionally, the dynamic leash may be adapted to be secured at one end to the occlusion device and at an opposing end to a wall of the patient's stomach.

Alternatively or additionally, the dynamic leash may be adapted to be secured at one end to the occlusion device and at an opposing end to an anti-migration anchor.

Another example may be found in a dynamic leash adapted for use in a gastric bypass system including an occlusion device and a tether secured to the occlusion device, the dynamic leash adapted to extend within a patient's stomach between an occlusion device adapted to be deployed relative to a patient's pylorus and a securement point. The dynamic leash includes an elastic member having a first end adapted to be secured relative to the occlusion device and a second end adapted to be secured to a securement point, where the elastic member dynamically opposes forces placed on the occlusion device by the tether.

Alternatively or additionally, the securement point may include an anastomosis anchor.

Alternatively or additionally, the securement point may include a wall of the patient's stomach.

Alternatively or additionally, the elastic member may include a spring.

Alternatively or additionally, the elastic member may include a pneumatic tube.

Alternatively or additionally, the elastic member may be adapted to resist gastrointestinal system movements that could otherwise cause the occlusion device to move into the patient's duodenum.

Another example may be found in an assembly adapted for use in a gastric bypass system including an occlusion device. The assembly includes a first gastric clip adapted to be secured to one side of a patient's stomach and a second gastric clip adapted to be secured to an opposing side of the patient's stomach. A first securement member is adapted to be secured to the first gastric clip. A second securement member is adapted to be secured to the second gastric clip. The first securement member and the second securement member releasably engage the occlusion device in order to limit distal movement of the occlusion device when the patient's pyloric sphincter expands.

Alternatively or additionally, the first securement member may include a first elastic band that is adapted to also be secured to the second gastric clip.

Alternatively or additionally, the second securement member may include a second elastic band that is adapted to also be secured to the first gastric clip.

Alternatively or additionally, the first securement member may include a first hook or bumper.

Alternatively or additionally, the second securement member may include a second hook or bumper.

Another example may be found in a device adapted to create an anastomosis and deliver a gastric bypass system in a single step. The device includes an electrocautery tip adapted to create an anastomosis, and a device segment adapted to releasably hold an anastomosis anchor in a collapsed configuration. The device segment is adapted to deploy the anastomosis anchor within the anastomosis, the anastomosis anchor expanding to an expanded configuration upon deployment.

Alternatively or additionally, a gastric bypass device may be delivered through a pull-back process.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a schematic view of a portion of a person's gastrointestinal (GI) system;

FIG. 2 is a schematic view of an illustrative gastric bypass system disposed within the GI system shown in FIG. 1, the illustrative gastric bypass system including an occlusion device, an anastomosis anchor, a tether and a dynamic leash;

FIGS. 3 through 26 are schematic views of illustrative occlusion devices usable in the illustrative gastric bypass system of FIG. 2;

FIGS. 27 through 32B are schematic views of illustrative anastomosis anchors and rings usable in the illustrative gastric bypass system of FIG. 2;

FIGS. 33 through 47 are schematic views of illustrative tethers usable in the illustrative gastric bypass system of FIG. 2, some of which are displayed in combination with occlusive devices;

FIG. 48 is a schematic view of a portions of a person's gastrointestinal (GI) system;

FIGS. 49 through 53D are schematic views of illustrative tethers usable in the illustrative gastric bypass system of FIG. 2 that protect the Papilla of Vater;

FIGS. 54 through 59 are schematic views of dynamic leashes usable in the illustrative gastric bypass system of FIG. 2;

FIGS. 60 and 61 are schematic views of secondary engagement apparatuses; and

FIG. 62 is a schematic view of a device that integrates anastomosis creation and gastric bypass system delivery; and

FIG. 63 is a schematic view of the resulting anastomosis and gastric bypass system.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some cases, the term “distal” refers to moving farther into the gastrointestinal system and the term “proximal” refers to moving out of the gastrointestinal system. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

This document relates to devices and methods for the medical treatment of conditions such as obesity and metabolic diseases. For example, this document provides methods and devices for bypassing portions of the GI tract to reduce nutritional update, decrease weight, and/or improve diabetes control.

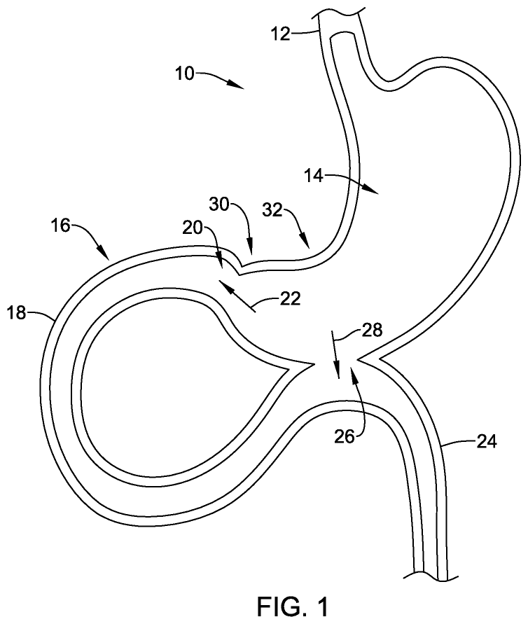

FIG. 1 is a schematic view of a portion of a human digestive tract 10. The digestive tract 10 includes an esophagus 12, a stomach 14, and a small intestine 16. The esophagus 12 connects the mouth to the stomach 14 and passes food to the stomach 14. The stomach 14 secretes digestive enzymes and gastric acid to aid in food digestion. The small intestine 16 is the organ where most of the absorption of nutrients and minerals from food takes place. The small intestine 16 includes a duodenum 18, a jejunum 24, and an ileum (not shown). A pyloric sphincter 20 controls a passage for movement 22 of partially digested food from the stomach 14 into the duodenum 18, which may be about 25-38 centimeters (cm) long. Food then passes to the jejunum 24, which may be about 2.25-2.75 meters (m) long. It will be appreciated that these dimensions are merely illustrative, and may vary from patient to patient.

An anastomosis 26 may be created between the stomach 14 and the small intestine 16. In some instances, the anastomosis 26 may be created between the stomach 14 and the duodenum 18. In some cases, the anastomosis 26 may be created between the stomach 14 and the jejunum 24. As an example, the anastomosis 26 may be created by a gastrojejunostomy. The anastomosis 26 can allow for movement 28 of food from the stomach 14 directly to the jejunum 24, bypassing the duodenum 18. In some cases, the anastomosis 26 can include a stent, staples, magnets, balloons, or other structure for maintaining the opening and connection between the stomach 14 and the small intestine 16. In some cases, the anastomosis 26 may be about 1-4 cm in diameter. In some cases, the stomach 14 may be considered as including a pylorus 30 that is positioned just upstream of the pyloric sphincter 20. The pylorus 30 may be considered as having a diameter that is greater than that of the pyloric sphincter 20. The stomach 14 may be considered as including an antrum 32 that is positioned just upstream of the pylorus 30. The antrum 32 may be considered as having a diameter that is greater than that of the pylorus 30.

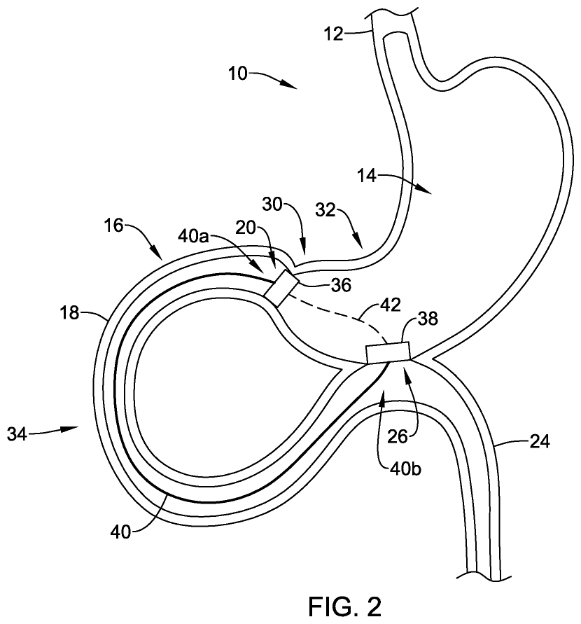

FIG. 2 schematically shows an illustrative gastric bypass device 34 shown disposed within the anatomy 10. The gastric bypass device 34 includes an occlusion device 36, which may be adapted to be placed within the pyloric sphincter 20, or within the pylorus 30 or within the antrum 32, depending on a desired degree of occlusion for the stomach 14. In some instances, the occlusion device 36 may be adapted to be placed within the antrum 32. The gastric bypass device 34 includes an anastomosis anchor 38 that may be adapted to be secured relative to the anastomosis 26. In some cases, the anastomosis anchor 38 may be adapted to be secured to an anastomosis structure (not shown) that may be present within the anastomosis 26 in order to preserve and hold together the anastomosis 26.

Because the occlusion device 36 is a foreign object, the stomach 14 may attempt to push the occlusion device 36 out of the pyloric sphincter 20 and down into the duodenum 18. The stomach 14 may attempt to push the occlusion device 36 out of the pyloric sphincter 20 and back into the stomach 14 itself. A tether 40 extends through the duodenum 18, and is secured at a first end 40a of the tether 40 to the occlusion device 36 and is secured at a second end 40b of the tether 40 to the anastomosis anchor 38. The tether 40 may be adapted to help hold the occlusion device 36 in place at its desired implantation location, against movement caused by the stomach 14 attempting to dislodge the occlusion device 36. In some cases, a dynamic leash 42 extends between the occlusion device 36 and the anastomosis anchor 38 and may be adapted to help hold the occlusion device 36 at its desired position. In some cases, the dynamic leash 42 may extend from the occlusion device 36 to an anchor position within a wall of the stomach 14. In some cases, the occlusion device 36 may include one or more anti-migration features such as hooks or tines, or perhaps a high friction coating over at least part of the occlusion device 36. In some cases, the anastomosis anchor 38 may include additional anti-migration features as well.

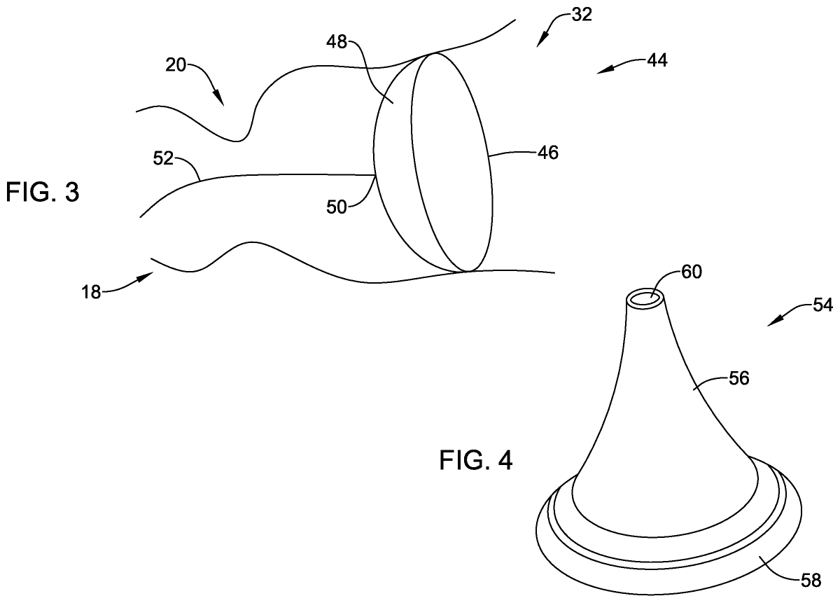

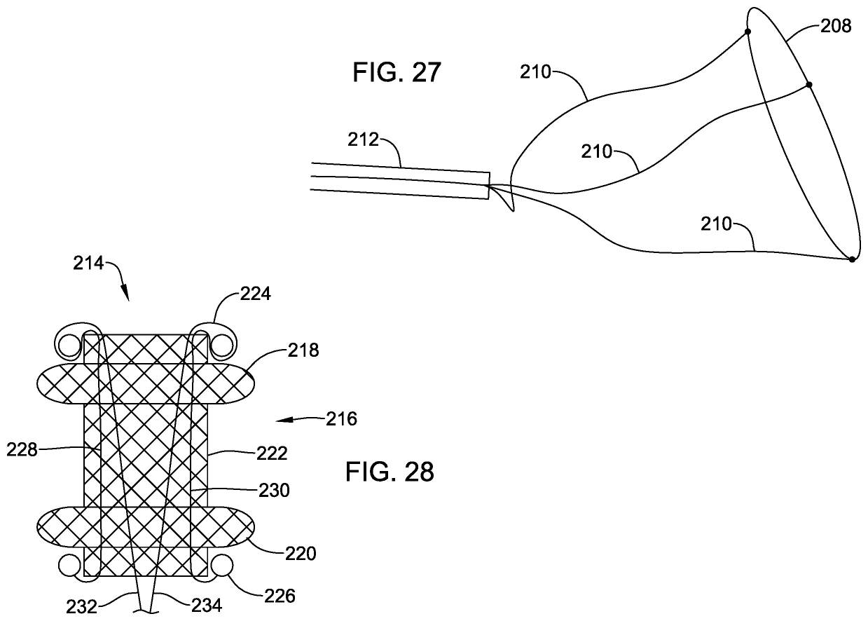

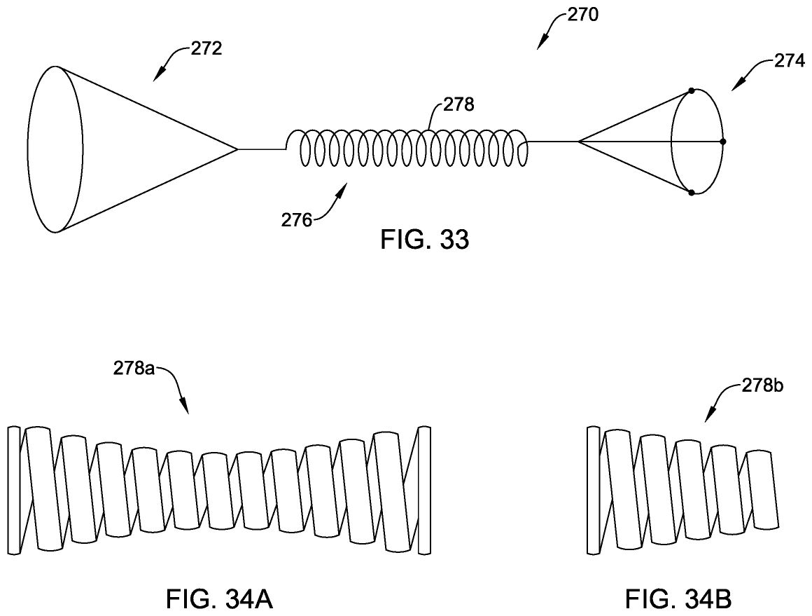

The gastric bypass device 34 is shown schematically because each component of the gastric bypass device 34, including the occlusion device 36, the anastomosis anchor 38, the tether 40 and the dynamic leash 42 may take a variety of different forms. FIGS. 3 through 26 provide illustrative but non-limiting examples of possible occlusion devices 36. FIGS. 27 through 32B provide illustrative but non-limiting examples of possible anastomosis anchors 38. FIGS. 33 through 53D provide illustrative but non-limiting examples of possible tethers 40. FIGS. 54 through 59 provide illustrative but non-limiting examples of possible dynamic leashes 42. It will be appreciated that a gastric bypass device such as the gastric bypass device 34 may include any of the occlusion devices 36, any of the anastomosis anchors 38, any of the tethers 40 and any of the dynamic leashes 42.

In some cases, an occlusion device may be disposed within or upstream of the pyloric sphincter 20. In some cases, depending on how much of the stomach 14 the physician or other professional wishes to occlude, the occlusion device may be disposed within the pylorus 30. The occlusion device 36 may be disposed within, or may extend to, the antrum 32. FIG. 3 through FIG. 26 provide examples of illustrative occlusion devices that may be used as the occlusion device 36 as part of the gastric bypass device 34.

FIG. 3 is a schematic view of an illustrative occlusion device 44 shown disposed near the antrum 32. The occlusion device 44 includes an annular ring 46 that is dimensioned to span the anatomy. It will be appreciated that the annular ring 46 may be dimensioned to help locate the occlusion device 44 at a desired position within the anatomy. For example, if there is a desire to locate the occlusion device 44 within the pyloric sphincter 20, the annular ring 46 may have an overall diameter of 1 to 3 cm. If there is a desire to locate the occlusion device 44 within the pylorus 30, the annular ring 46 may have an overall diameter of 2 to 8 cm. If there is a desire to locate the occlusion device 44 within the antrum 32, the annular ring 44 may have an overall diameter of 4 to 12 cm.

The occlusion device 46 includes a tapered body 48 that tapers from the annular ring 46 (which may be considered as defining the maximum outer diameter of the occlusion device 44) to a minimum diameter endpoint 50. The minimum diameter endpoint 50 may be adapted to be secured to a tether 52, for example. The tapered body 48 may taper smoothly from its maximum outer diameter to its minimum outer diameter. The tapered body 48 may taper in a step-wise fashion, with one or more abrupt diameter changes. In some cases, the tapered body 48 may have a curved profile. The tapered body 48 may be adapted to prevent materials such as food, chyme and other gastric contents from flowing through the tapered body 48. In some cases, the tapered body 48 may be constructed out of an impervious material such as but not limited to a polymeric material. In some cases, the tapered body 48 may include a polymeric membrane disposed over some sort of support frame (not shown).

The thickness, durometer and lubricity of the polymeric material used to form the occlusion device 54 may vary along a length of the occlusion device 44. The occlusion device 44 may have a funnel shape, for example, or a cyclone shape. The occlusion device 44 may have a hemispherical or even a spherical shape. The occlusion device 44 may include an indentation (not shown) to accommodate a support ring. In some cases, the occlusion device 44 may be collapsible in order to aid deliverability. The occlusion device 44 may include a membrane or other covering spanning the opening defined by the annular ring 46 in order to keep materials from accumulating within the occlusion device 44.

The occlusion device 44 may be formed of any suitable polymeric or metallic material, as long as that material is adapted for long-term survival in the gastric environment. In some cases, the occlusion device 44 may be formed of silicone or another polymer. The occlusion device 54 may be formed via 3D printing, for example. In some cases, the occlusion device 54 may be molded or even e-spun.

In some cases, the occlusion device 44 may include additional metallic supports (not shown) in order to help provide an outward radial force to better engage the anatomy. In some cases, the material used to form the occlusion device 44 may be thicker near the annular ring 46. The occlusion device 44 may be formed of a shape memory material that allows the occlusion device 44 to have a remembered configuration for deployment, and to be able to temporarily be deformed from the remembered configuration during delivery. While not shown, the occlusion device 44 may include anchors such as outward prongs, hooks, splines or tines. The occlusion device 44 may include a surface treatment that encourages endothelization. These are just examples.

FIG. 4 is a schematic view of an illustrative occlusion device 54 that may be considered as being an example of the occlusion device 44. The illustrative occlusion device 54 is formed of a single continuous polymeric body 56 that extends from an annular ring 58 representing a maximum outer diameter of the occlusion device 54 to a minimum diameter endpoint 60. The minimum diameter endpoint 60 may be considered as being adapted to be secured to a tether such as the tether 40. The annular ring 58 may be dimensioned to locate the occlusion device 54 in a desired location relative to the pyloric sphincter 20, the pylorus 30 or the antrum 32, for example.

The occlusion device 54 may be considered as being deformable and endoscopically deliverable. The annular ring 56 is adapted to exert an outward radial force in order to engage the anatomy. If the occlusion device 54 is intended for deployment within the pyloric sphincter 20, the annular ring 58 may have an overall diameter of 1 to 3 cm. If the occlusion device 54 is intended for deployment within the pylorus 30, the annular ring 58 may have an overall diameter of 2 to 8 cm. If the occlusion device 54 is intended for deployment within the antrum 32, the annular ring 58 may have an overall diameter of 4 to 12 cm.

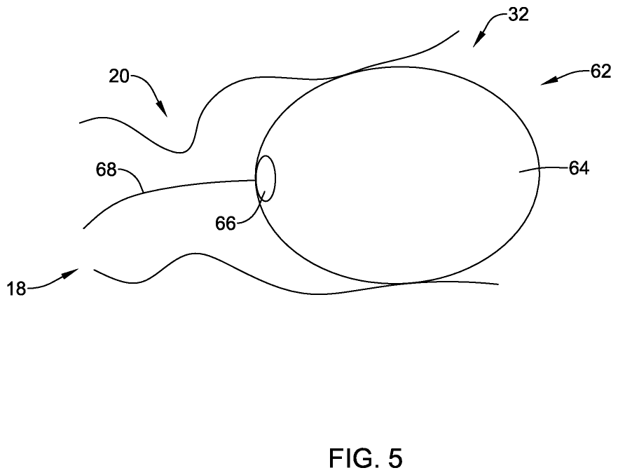

FIG. 5 is a schematic view of an illustrative occlusion device 62 shown disposed near the antrum 32. The occlusion device 62 has an inflatable body 64 that can be filled with a gas or other fluid such as saline in order to hold its inflated shape (as shown). The inflatable body 64 may be an inflatable balloon, for example. In some cases, the occlusion device 62 may be delivered with the inflatable body 64 in a deflated configuration. Once the occlusion device 62 reaches its desired deployment location, the inflatable body 64 may be filled with gas or other fluid, or perhaps a gel, in order to expand to its expanded configuration (as shown). The inflatable body 64 may have a spherical or semispherical shape, for example, and may have a maximum outer diameter that helps to locate the occlusion device 62 relative to the anatomy. If the occlusion device 62 is intended for deployment within the pyloric sphincter 20, the inflatable body 64 may have a maximum diameter of 1 to 3 cm. If the occlusion device 62 is intended for deployment within the pylorus 30, the inflatable body 64 may have a maximum diameter of 2 to 8 cm. If the occlusion device 62 is intended for deployment within the antrum 32, the inflatable body 64 may have a maximum diameter of 4 to 12 cm.

The occlusion device 62 may be formed of any material such as a polymeric material that is able to withstand the highly acidic gastric environment. As an example, the occlusion device 62 may be formed of silicone, although the occlusion device 62 may include additional fiber reinforcements. The occlusion device 62 may be formed via 3D printing, for example. The occlusion device 62 may be molded or e-spun. The occlusion device 62 may be formed via dip coating. As another example, the occlusion device 62 may be formed by e-spinning two halves, then dip-coating the two halves together to form the occlusion device 62. The occlusion device 62 includes an attachment point 66 that is adapted to be secured to a tether 68.

In some cases, the inflatable body 64 may have a stiffness profile that can vary. The inflatable balloon 64 may be relatively flexible, which allows the walls of the inflatable body 64 to compress and expand with peristalsis. The inflatable balloon 64 may be relatively stiff, thereby helping to anchor the occlusion device 62 in position relative to the antrum 32. In some cases, the occlusion device 62 may have a variety of different shapes. The occlusion device 62 may have a three dimensional funnel shape. The occlusion device 62 may have a hemispherical top, or may not have a hemispherical top. The occlusion device 62 may have an undefined, organic shape. The occlusion device 62 may include one or more protruding lips or rings that help secure the occlusion device 62 in place relative to the anatomy. While not shown, the occlusion device 62 may include anchors such as outward prongs, hooks, splines or tines. The occlusion device 62 may include a surface treatment that encourages endothelization.

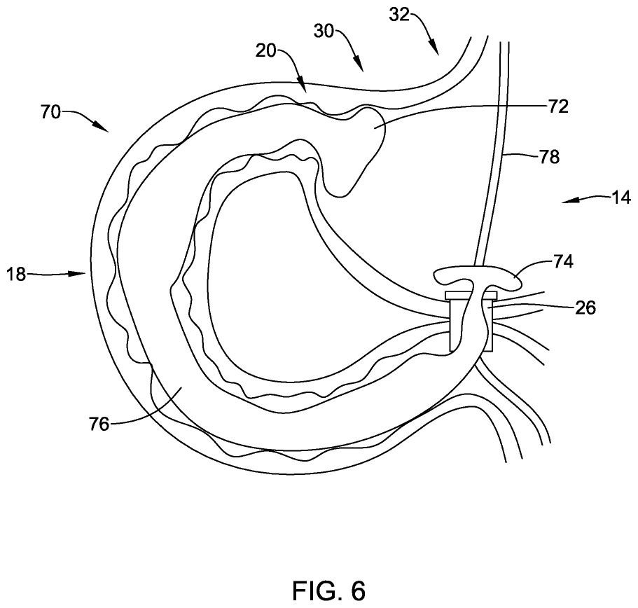

While shown deployed within the antrum 32, this is not required in all cases. For example, the occlusion device 62 may extend through the antrum 32 and partially into the duodenum 18. In some cases, as shown in FIG. 6, the occlusion device 62 may extend from the antrum 32, through the pyloric sphincter 20, through the entirety of the duodenum 18 and up through the anastomosis 26. Accordingly, the occlusion device 62 may act as occlusion device, tether and anastomosis anchor. FIG. 6 shows an occlusion device 70 having a first end 72 that is located near the pyloric sphincter 20 and a second end 74 that extends through the anastomosis 26. The occlusion device 70 includes an elongate inflatable body 76 that extends through the duodenum 18 from the first end 72 of the occlusion device 70 to the second end 74 of the occlusion device 70. As shown, a deployment feature 78 extends into the stomach 14 and to the second end 74 of the occlusion device 70.

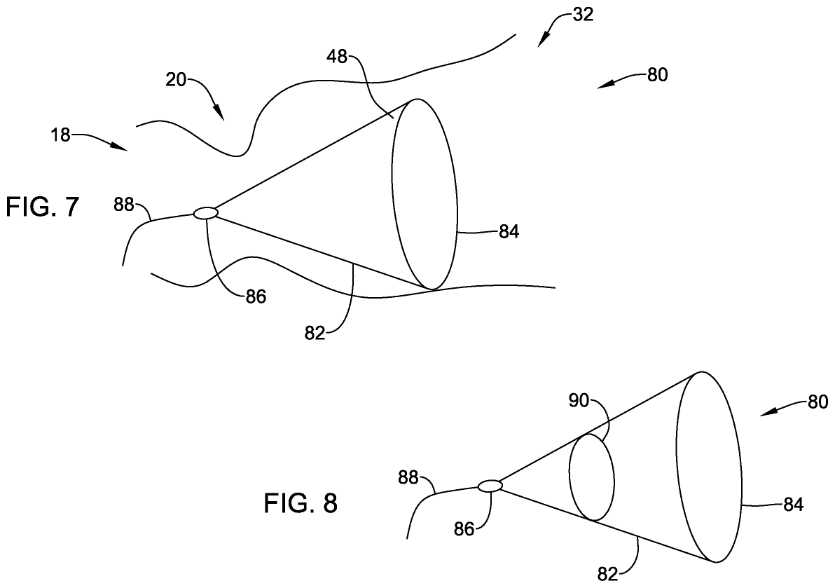

FIG. 7 is a schematic view of an illustrative occlusion device 80 that may be considered as being an example of the occlusion device 44. The illustrative occlusion device 80 is shown within the anatomy, and is shown near the antrum 32. In some cases, the occlusion device 80 may occlude from 10 percent to 50 percent of the stomach 14, and may conform to the wall of the stomach 14. The occlusion device 80 includes a thin membrane funnel 82 that is funnel-shaped or conical. The thin membrane funnel 82 may be formed of silicone or expanded polytetrafluoroethylene (e-PTFE), for example. The thin membrane funnel 82 may be formed of a polyurethane that is highly resistant to acids and chemicals. In some cases, a low molecular weight resin such as that available from Cray Valley under the Krasol name may be mixed into a polyurethane elastomer that is highly chemically resistant. In some cases, such polybutadiene-urethanes have a rubber character, exceptional resistance to hydrolysis and chemicals, good elasticity and may be reinforced using common rubber fillers.

The occlusion device 80 extends from an annular ring 84 that represents a maximum outer diameter of the occlusion device 80 to a minimum diameter endpoint 86. The minimum diameter endpoint 86 may be considered as being adapted to secure to a tether 88. The large end of the occlusion device 80 may be covered or uncovered. The annular ring 84, which may be a support ring added to the occlusion device 80, may be dimensioned to locate the occlusion device 80 in a desired location relative to the pyloric sphincter 20, the pylorus 30 or the antrum 32, for example. If the occlusion device 80 is intended for deployment within the pyloric sphincter 20, the annular ring 84 may have an overall diameter of 1 to 3 cm. If the occlusion device 80 is intended for deployment within the pylorus 30, the annular ring 84 may have an overall diameter of 2 to 8 cm. If the occlusion device 80 is intended for deployment within the antrum 32, the annular ring 84 may have an overall diameter of 4 to 12 cm.

The annular ring 84 may be adapted to exert an outward radial force to help hold the occlusion device 80 in position relative to the anatomy. The occlusion device 80 may include partial or entire fiber or metallic reinforcements such as ultra high weight polyethylene (UHMWPE) or Nitinol. The occlusion device 80 may be manufactured by attaching the thin membrane funnel 82 to the annular ring 84 via sewing, suturing, thermal bonding or chemical bonding, for example.

In some cases, as shown in FIG. 8, the occlusion device 80 may include a second, intermediate support ring 90 that helps to support the thin membrane funnel 82. The occlusion device 80 may include a third support ring, a fourth support ring, and so on. The intermediate support ring 90 (and a support ring added to the annular ring 84) may be formed of a shape memory metal such as a nickel-titanium alloy, including Nitinol. While not shown, the occlusion device 80 may include anchors such as outward prongs, hooks, splines or tines. The occlusion device 80 may include a surface treatment that encourages endothelization.

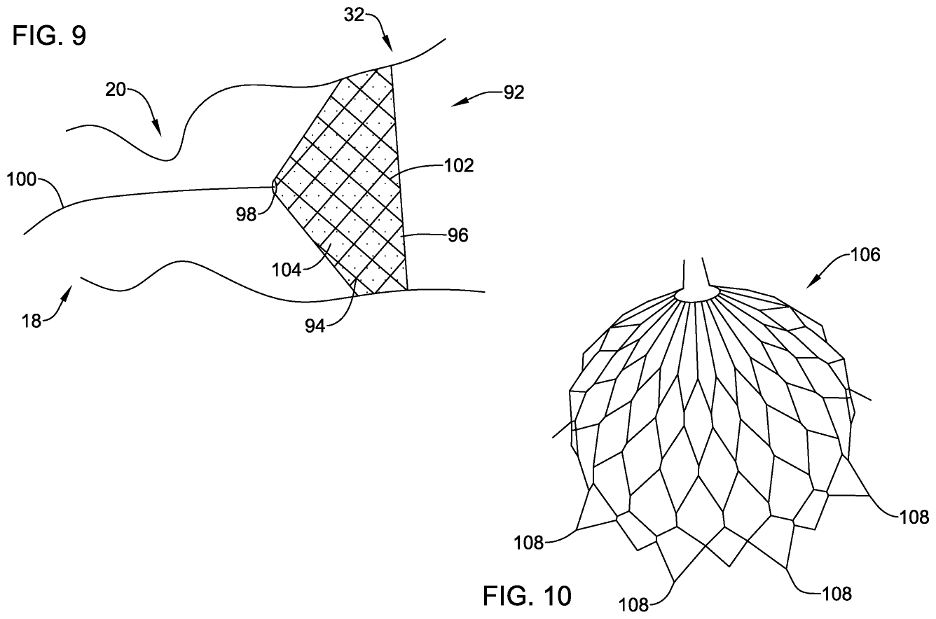

FIG. 9 is a schematic view of an illustrative occlusion device 92. The illustrative occlusion device 92 has a structured frame 94 that extends from a maximum diameter opening 96 to a minimum diameter endpoint 98. The minimum diameter endpoint 98 is adapted to be secured to a tether 100. In some cases, the maximum diameter opening 96 may include a covering (not shown) that spans the opening. The covering, if included, may be concave or convex.

The maximum diameter opening 96 may be dimensioned to locate the occlusion device 92 in a desired location relative to the pyloric sphincter 20, the pylorus 30 or the antrum 32, for example. If the occlusion device 92 is intended for deployment within the pyloric sphincter 20, the maximum diameter opening 96 may have an overall diameter of 1 to 3 cm. If the occlusion device 92 is intended for deployment within the pylorus 30, the maximum diameter opening 96 may have an overall diameter of 2 to 8 cm. If the occlusion device 92 is intended for deployment within the antrum 32, the maximum diameter opening 96 may have an overall diameter of 4 to 12 cm.

The structured frame 94 may be woven or braided. In some cases, the structured frame 94 may be a laser cut structure. As shown, the structured frame 94 has a number of individual struts 102 that are connected to provide rigidity to the structured frame 94. The structured frame 94 is adapted to have shape retention such that the structured frame 94 reverts to an expanded configuration (as shown) subsequent to being compressed or otherwise compressed for delivery. The dimensions of the individual struts 102 may be varied to provide particular properties to the structured frame 94. The structured frame 94 may have a cone shape or a funnel shape. The structured frame 94 may be spherical or hemispherical in shape. In some cases, the structured frame 94 may be formed from two or more different parts that are secured together. In some cases, the structured frame 94 may be formed from a laser-cut, expandable tube. The structured frame 94 may be a multi-fiber braided or woven structure. The structured frame 94 may be formed from discrete wires that are soldered, welded or otherwise joined together to form the structured frame 94. The structured frame 94 may be cast from molten metal, for example.

The occlusion device 92 includes a covering or coating 104 (shown in a dotted pattern) that covers at least a portion of the structured frame 94. The covering or coating 104 may be PTFE or e-PTFE. The covering or coating 104 may be silicone or another chemically resistant polymer. The covering or coating 104 may be applied via dip coating, spray coating or e-spinning, for example. While not shown, the occlusion device 92 may include anchors such as outward prongs, hooks, splines or tines. The occlusion device 92 may include a surface treatment that encourages endothelization.

FIG. 10 is a schematic view of an illustrative structured frame 106 that may be considered as an example of the structured frame 94. The structured frame 106 includes a number of outward-facing tines 108 that help to anchor the structured frame 106 (and hence the occlusion device including the structured frame 106) in position within the anatomy. When included as part of an occlusion device, the structured frame 106 would include a coating or covering such as the coating or covering 104 shown in FIG. 9.

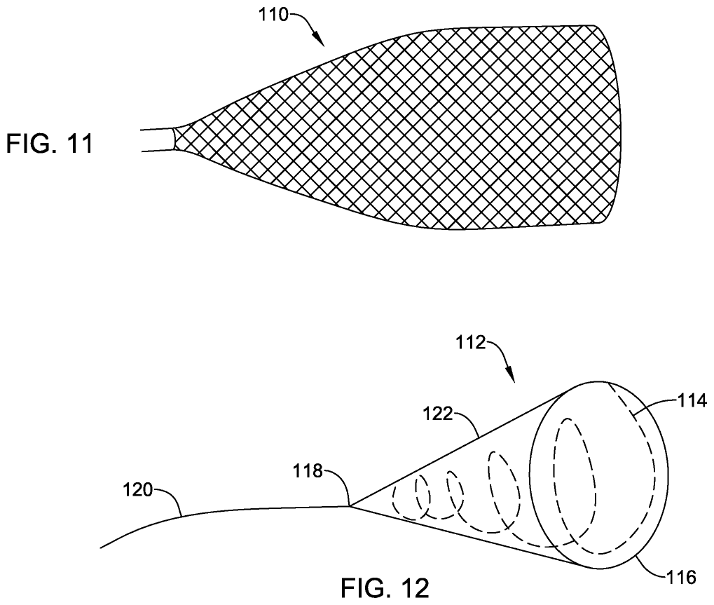

FIG. 11 is a schematic view of an illustrative structured frame 110 that may be considered as an example of the structured frame 94. The structured frame 110 is an example of a braided structure. When included as part of an occlusion device, the structured frame 106 would include a coating or covering such as the coating or covering 104 shown in FIG. 9.

FIG. 12 is a schematic view of an illustrative occlusion device 112. The illustrative occlusion device 112 includes a coiled support wire 114 that extends from a maximum diameter end 116 to a minimum diameter endpoint 118 that is adapted to be secured to a tether 120. The coiled support wire 114 supports a membrane 122 that covers the coiled support wire 114, thereby occluding stomach contents from passing through the occlusion device 112 and into the duodenum 18. In some cases, the coiled support wire 114 is formed of a shape memory material such as Nitinol.

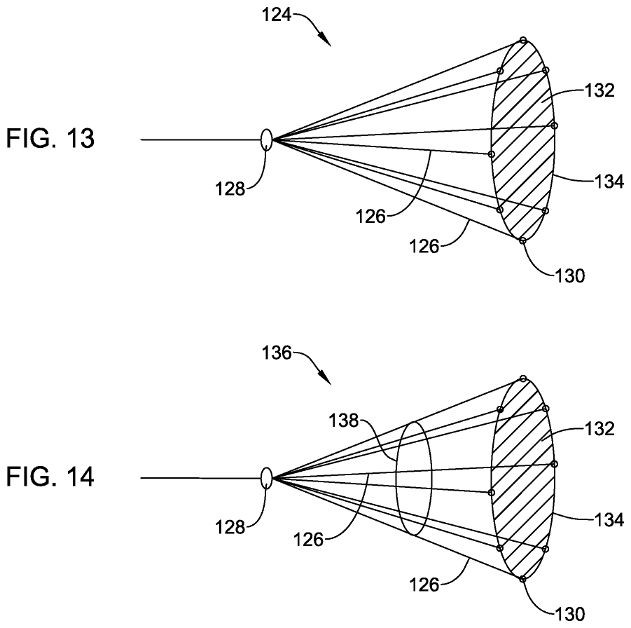

FIG. 13 through FIG. 16 are schematic views of illustrative occlusion devices that include radial support members supporting an occlusion covering. In some cases, the radial support members are incompressible in order to ensure occluder covering engagement. The support members are adapted to enable better self-alignment and engagement of the occluder covering. The occlusion devices have an open structure that allows any chyme that escapes past the occluder covering to pass through the pylorus. The occluder covering exerts an outward radial force to help anchor the occlusion devices relative to the anatomy.

FIG. 13 is a schematic view of an illustrative occlusion device 124. The illustrative occlusion device 124 includes a number of radial support members 126 that extend from a starting point 128 to a terminal end 130, where the terminal end 130 defines a maximum outer diameter of the occlusion device 124. In some cases, as shown, the occlusion device 124 includes an occluder covering 132 located at the terminal end 130.

The radial support members 126 can be metal or polymer. In some cases, the radial support members 126 are a shape memory metal such as Nitinol. The radial support members 126 may be wrapped or bent to enable reduced dimensions for deliverability. The radial support members 126 may sit within the pylorus 30 or even extend into the duodenum 18 in order to reduce possible trauma to the pyloric sphincter 20. While not shown, the radial support members 126 may be covered with a membrane or other material, thereby forming a cone shape. In some cases, an opening of the cone may also be covered with a membrane or other material.

The occlusion disk 132 may be formed of a polymer such as silicone, ePTFE or a fabric or metallic mesh. In some cases, the occlusion disk 132 may include a support ring 134. The support ring 134, if included, may be polymeric or metallic. In some cases, the support ring 134 may be formed of Nitinol. While not shown, the occlusion disk 132 may include outward facing prongs, hooks, splines or tines in order to help engage tissue and thus help anchor the occlusion device 124 in position.

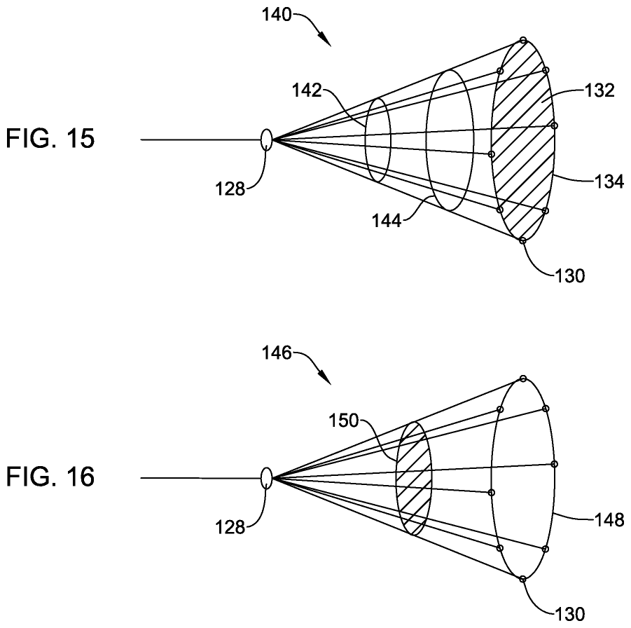

FIG. 14 is a schematic view of an illustrative occlusion device 136. The illustrative occlusion device 136 is similar to the occlusion device 124, but includes a support ring 138 that is located intermediate between the starting point 128 and the terminal end 130. FIG. 15 is a schematic view of an illustrative occlusion device 140. The illustrative occlusion device 136 is similar to the occlusion device 124, but includes both a first support ring 142 and a second support ring 144.

FIG. 16 is a schematic view of an illustrative occlusion device 146. The illustrative occlusion device 146 is similar to the occlusion devices 124, 136 and 140, but represents a bit of a rearrangement. The radial support members 126 extend to an anchor ring 148 that is formed at the terminal end 130. The anchor ring 148 is adapted to secure the occlusion device 146 in position within the anatomy. The occlusion device 146 includes an occlusion disk 150 that is positioned intermediate the starting point 128 and the terminal end 130.

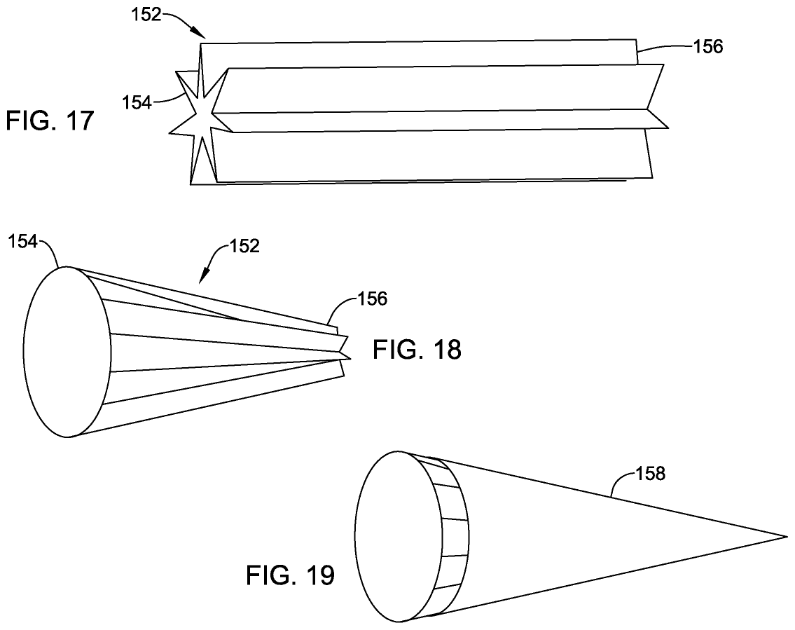

FIG. 17 through FIG. 19 are schematic views of occlusion devices that are made from, or otherwise include a corrugated tube. The corrugated tube can be formed of any suitable polymeric or metallic material, and the corrugation may be collapsible such that the occlusion devices including corrugated tubes can be endoscopically deliverable.

FIG. 17 shows an occlusion device 152 in a collapsed configuration while FIG. 18 shows the occlusion device 152 in an expanded configuration. The occlusion device 152 extends from a first end 154 to a second end 156. As shown in FIG. 18, the first end 154 defines the maximum outer diameter portion of the occlusion device 152 while the second end 156 defines the minimum outer diameter portion of the occlusion device 152, and is adapted to extend through the pyloric sphincter 20 with the second end 156 facing an interior of the stomach 14.

The corrugation may extend the length of the occlusion device 152. In some cases, as shown in FIG. 19, the corrugation may only form the second end 156, with a membrane filter 158 extending distally from the second end 156. The corrugation may be a solid material or a corrugated frame with an atraumatic covering. The corrugation may be a tube that is constrained at one end to form a funnel. The corrugation may be designed as a funnel, with the corrugation depth varying along a length of the funnel. The corrugation may be formed of a shape memory metal or polymer. The corrugation may include additional metallic or non-metallic supports. The corrugation may have a thicker section at the second end 156. In some cases, a membrane may span the second end 156. The membrane may be polymeric, such silicone, or even a fabric. While not shown, the occlusion device 152 may include outward facing prongs, hooks, splines or tines in order to help engage tissue and thus help anchor the occlusion device 152 in position.

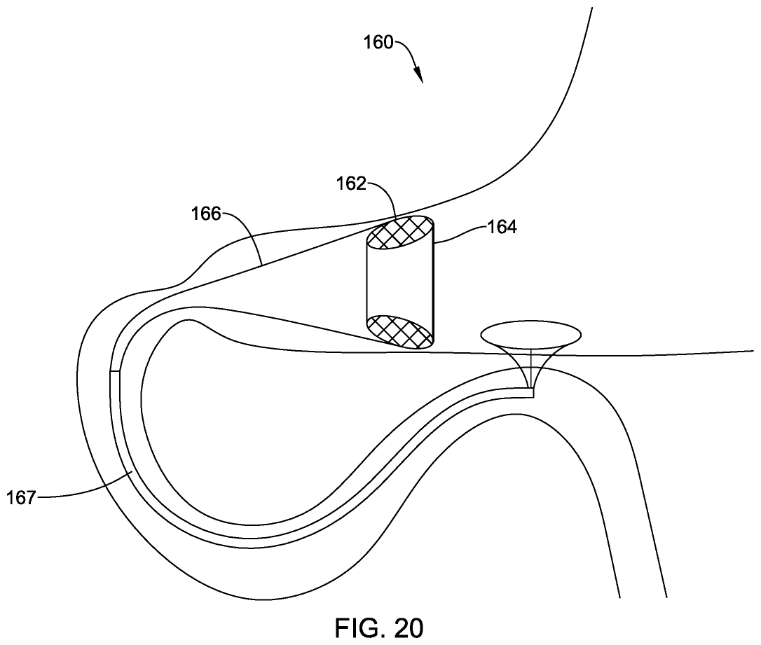

FIG. 20 through FIG. 23 are schematic views of occlusion devices that include a frame and a membrane. FIG. 20 shows an occlusion device 160 that includes a frame 162 and a membrane cap 164. A polymeric membrane 166 extends distally from the frame 162 and the membrane cap 164, and extends to a tether 167. The polymeric membrane 162 may be PTFE, ePTFE or silicone, for example. The frame 162 may be a laser-cut expandable tube, for example, or the frame 162 may be a multi-fiber braided structure. The frame 162 may be formed of Nitinol or stainless steel. In some cases, polymers or other metals may be used to form the frame 162. The frame 162 may include anchoring features such as outward facing prongs, hooks, splines or tines. The occlusion device 160 may include a coating that encourages endothelialization. The occlusion device 160 has an overall funnel or conical shape.

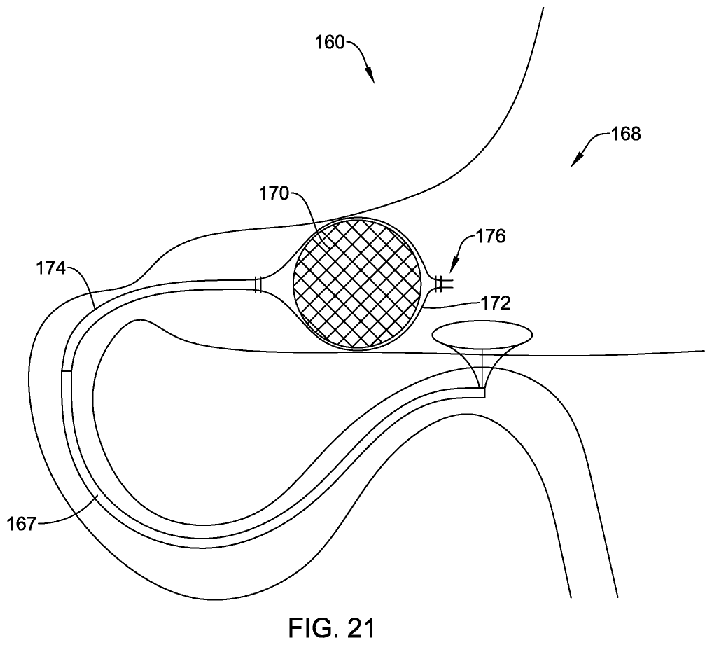

FIG. 21 shows an occlusion device 168 that includes a frame 170 and a membrane 172 covering the frame 170. The membrane 172 extends distally to a tubular member 174. In some cases, the membrane 172 may include a suture point 176. In some cases, the membrane 172 may be an integral member. The tubular member 174 is formed of a polymeric material such as ePTFE. The frame 170 is spherical in shape.

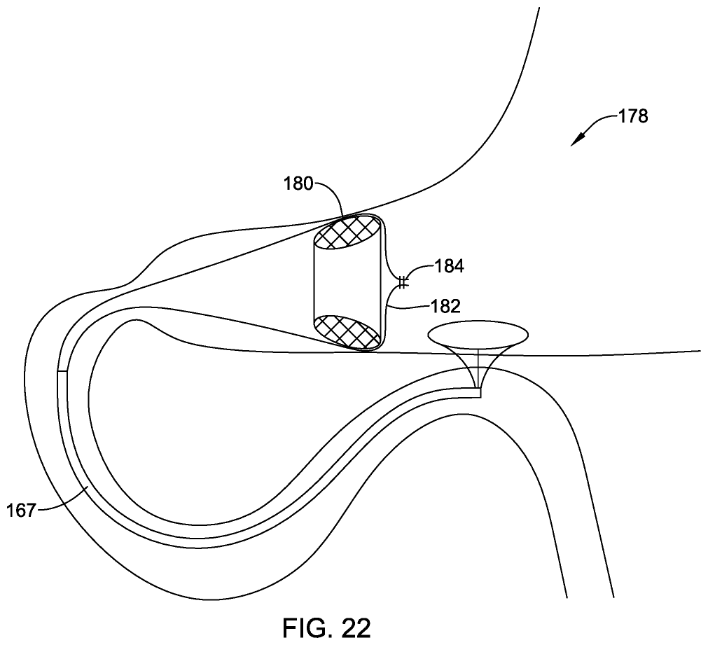

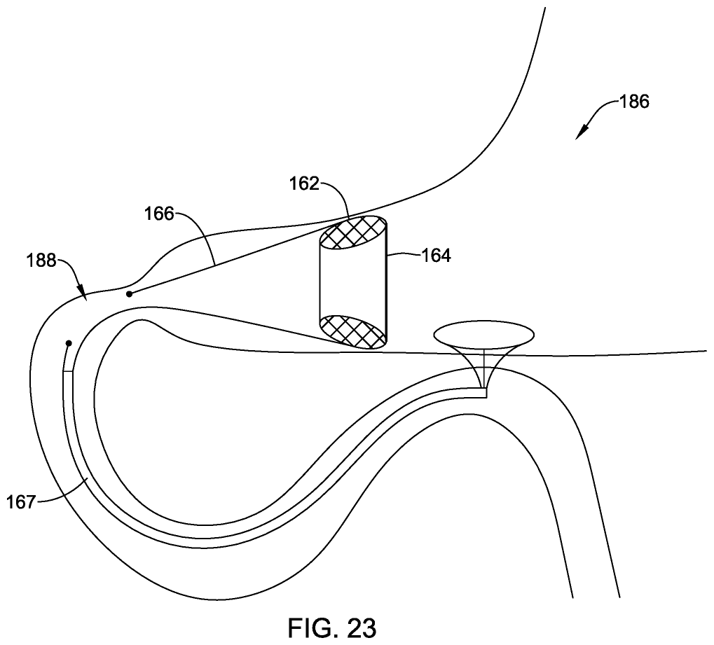

FIG. 22 is a schematic view of an occlusion device 178 that includes a frame 180 and a membrane 182. The membrane 182 envelops the frame 180, and extends distally from a suture point 184. The membrane 182 extends to a tether (not shown). FIG. 23 shows an occlusion device 186 that is similar to the occlusion device 160 (FIG. 20), but the polymeric membrane 166 includes an opening 188 that allows any otherwise-entrapped chyme to exit.

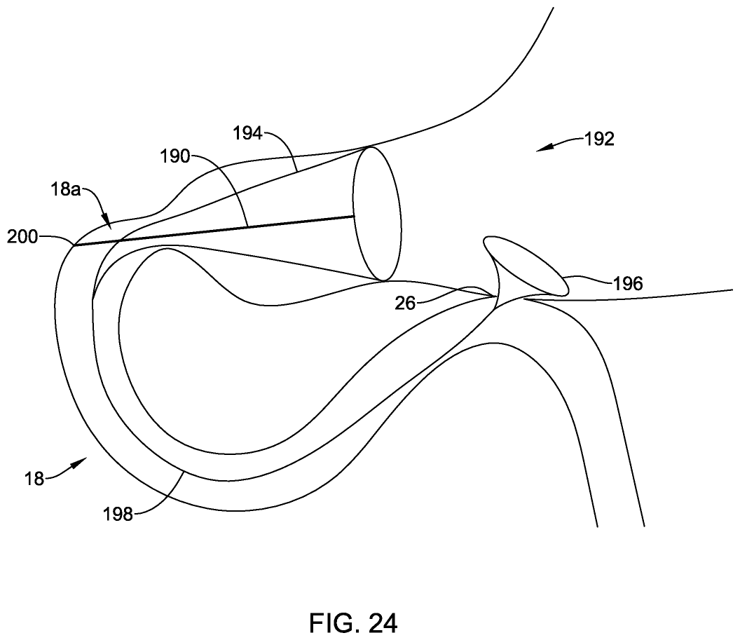

FIG. 24 through FIG. 26 are schematic views of occlusion devices that include a stiff feature as part of the occlusion device. The stiff feature may be adapted such that the stiff feature is unable to pass through a tortuous bend typically found in the proximal duodenum 18a. The proximal duodenum 18a is the part of the duodenum 18 that is just distal of the pyloric sphincter 20, and generally includes a tortuous bend. FIGS. 24 through 26 show a stiff feature 190 that may be incorporated into any of the occlusion devices described herein. FIGS. 24 through 26 show an illustrative gastric bypass device 192 including an occlusion device 194, an anastomosis anchor 196 and an intervening tether 198.

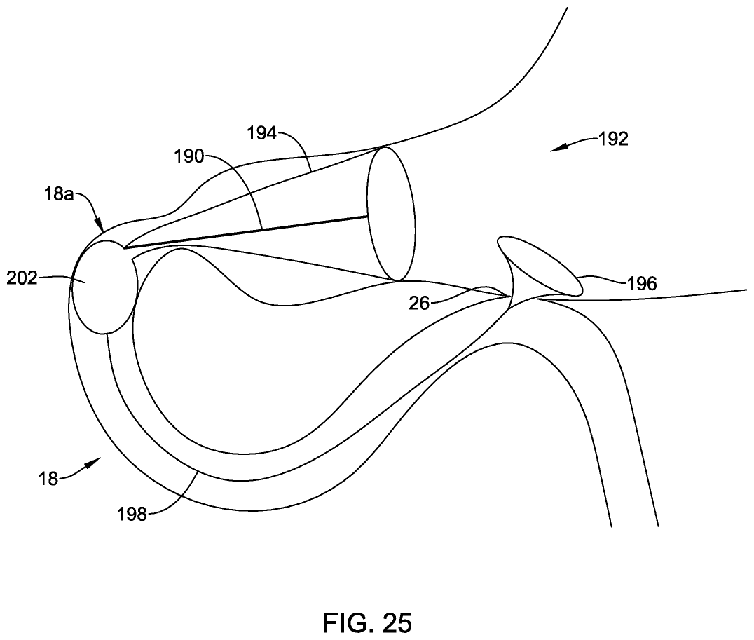

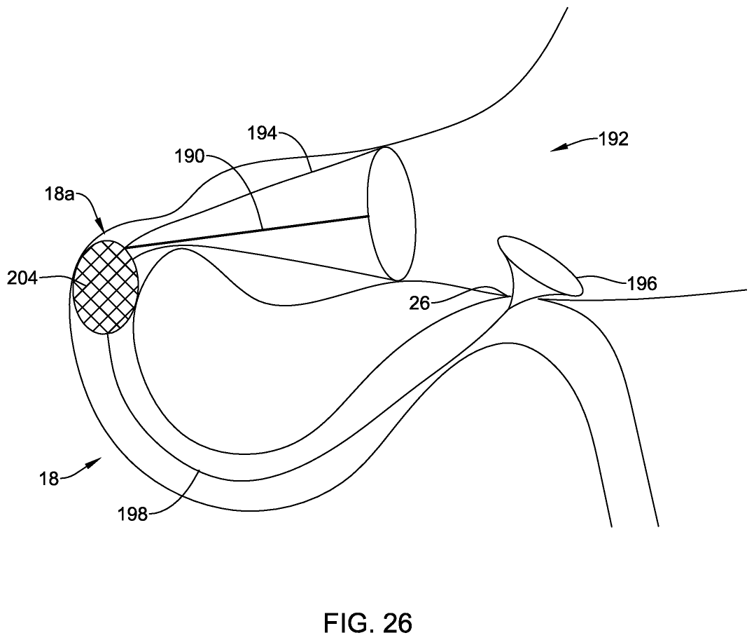

In FIG. 24, the stiff feature 190 includes a bumper 200 that in some instances may be considered as being an extension of the stiff feature 190. The bumper 200, in combination with the stiff feature 190, prevents distal movement of the occlusion device 194 because the bumper 200 cannot fit through the tortuous bend in the proximal duodenum 18a. In FIG. 25, the stiff feature 190 includes an inflatable bumper 202 that prevents both proximal and distal movement of the occlusion device 194. In FIG. 26, the stiff feature 190 includes a frame bumper 204. The frame bumper 204 is a rigid, self-expanding frame that prevents both proximal and distal movement of the occlusion device 194.

FIG. 27 through FIG. 32B provide examples of illustrative anastomosis anchors that may be used as the anastomosis anchor 38 as part of the gastric bypass device 34. In some cases, an anastomosis structure such as an expandable stent, a pair of magnetic structures, or the like, may be implanted proximate the anastomosis 26 in order to help hold the anastomosis 26 together. The anastomosis structure also provides something for an anastomosis anchor to be secured to.

FIG. 27 is a schematic view of an illustrative anastomosis anchor 206 that may be secured relative to the anastomosis 26 (FIG. 1). The corresponding anastomosis structure is not shown in FIG. 27, but it will be appreciated that one of the features of the anastomosis anchor 206 is that it has a diameter that is greater than a lumen diameter of the anastomosis structure. Accordingly, advancing the anastomosis anchor 206 proximally through the anastomosis 26 (and through the anastomosis structure) means that once the anastomosis anchor 206 has reached its expanded configuration (as shown), the anastomosis anchor 206 is not able to pull through the anastomosis 26 (or the anastomosis structure), thereby anchoring the anastomosis anchor 206 relative to the anastomosis 26 (and the anastomosis structure).

As shown in FIG. 27, the anastomosis anchor 206 includes a ring 208 that has an outer dimension that is greater than the lumen diameter of the anastomosis 26 (or the anastomosis structure). While shown as being annular, the ring 208 may take any of a variety of different shapes, such as circular or polygonal. The ring 208 may be concave or convex. The ring 208 may be regular or irregular in shape. The ring 208 may be formed of a material that is resistant to the highly acidic gastric environment. The ring 208 may be formed of a metal such as Nitinol or stainless steel. The ring 208 may be formed of a polymer such as PTFE or an ultra-high molecular weight polyethylene (UHMwPE) fiber available commercially under the Dyneena® name. The ring 208 may be a composite formed of several different materials. The ring 208 may be a wire that is joined with a coupler. The ring 208 may be a laser-cut structure. In some cases, the ring 208 may be a woven or braided structure. The ring 208 may include a coating or covering that is lubricious and/or resistant to corrosion.

The anastomosis anchor 206 includes a number of attachment members 210 that extend between the ring 208 and a tether 212. While a total of three attachment members 210 are shown, it will be appreciated that this is merely illustrative, as the anastomosis anchor 206 may include any number of attachment members 210. In some cases, having at least three attachment members 210 help to stabilize the position of the ring 208 relative to the anastomosis 26 (and the anastomosis structure). The attachment members 210 may be flexible and thread-like. The attachment members 210 may be rigid. While not shown, the ring 208 may instead be attached to the tether 212 via a polymeric membrane that spans from the ring 208 to the tether 212.

FIG. 28 is a schematic view of an illustrative anastomosis anchor 214 that is shown disposed relative to an illustrative anastomosis structure 216. The illustrative anastomosis anchor 214 may be considered as being an opposed dual-ring anchor. The illustrative anastomosis structure 216 includes a first annular section 218 that is adapted to be disposed within the stomach 14 and a second annular section 220 that is adapted to be disposed within the small intestine 16. In some instances, the second annular section 220 may be adapted to be disposed within the duodenum 18 or the jejunum 24. An intervening portion 222 extends between the first annular section 218 and the second annular section 220. It will be appreciated that the intervening portion 222 defines a lumen extending through the anastomosis structure 216. Accordingly, the dimensions of the intervening portion 222 define a minimum size for the ring 208 (of the anastomosis anchor 206 shown in FIG. 27). In some cases, the anastomosis structure 216 may be considered as being a self-expanding stent that is woven or braided. In some cases, the anastomosis structure 216 may be considered as being an example of the Axios® stent available commercially from Boston Scientific.

The anastomosis anchor 214 may include a first ring 224 that is adapted to be secured above the first annular section 218 of the anastomosis structure 216. The anastomosis anchor 214 may include a second ring 226 that is adapted to be secured below the second annular section 220 of the anastomosis structure 216. In this, terms such as above or below merely refer to the illustrated orientation. The anastomosis structure 216 could be deployed in any orientation, including an orientation that is largely upside down from what is shown in FIG. 28, for example.

The anastomosis anchor 214 includes one or more members 228 and 230 that extend between the first ring 224 and the second ring 226. The anastomosis anchor 214 also includes one or more connectors 232 and 234 that extend downward from the first ring 224 in order to couple the anastomosis anchor 214 with a tether. In some cases, a tensile force applied to the connectors 232 and 234 may result in a distance between the first ring 224 and the second ring 226 becoming reduced. As the first ring 224 and the second ring 226 becomes smaller, the resulting forces applied to the anastomosis structure 216 cause the anastomosis structure 216 to shorten in length and to grow radially. As the first annular section 218 and the second annular section 220 of the anastomosis structure 216 grow radially, the first annular section 218 and the second annular section 220 of the anastomosis structure 216 provides an enhanced engagement with the tissue, thereby helping to ensure no device migration.

In some cases, the one or more members 228 and 230 and/or the one or more connectors 232 and 234 may include one or more strings. The one or more members 228 and 230 and/or the one or more connectors 232 and 234 may be braided or coiled structures, or may be sheaths. The one or more members 228 and 230 and/or the one or more connectors 232 and 234 may be covered or uncovered, for example. Each of the components of the anastomosis anchor 214 may independently be made of materials that are resistant to the harsh gastric environment. Metals such as Nitinol and stainless steel may be used, as may polymers such as PTFE and ultra-high molecular weight polyethylenes (UHMwPE) fiber available commercially under the Dyneena® name. The connectors 232 and 234 may have a single attachment point to a tether, or may have multiple attachment points.

In some cases, parts or all of the anastomosis anchor 214 may be covered, with the proviso that the through-lumen through the anastomosis anchor 214 remains open so that food and chyme can pass through. The covering may serve to help protect parts or all of the anastomosis anchor 214 from the gastric environment. Coverings, if included, may reduce interactions with chyme or food particles. Coverings, if included, may reduce friction or interactions with the gastric environment tissue. Coverings could be tight-fitting or loose, and may be PTFE, ePTFE or other polymers. A covering, if included, could encapsulate largely the entire anastomosis anchor 214, or only individual components thereof.

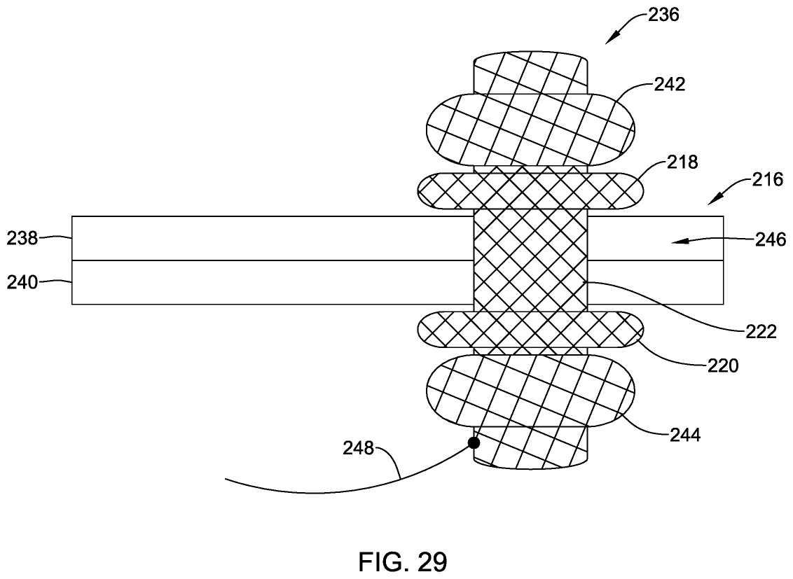

FIG. 29 is a schematic view of an illustrative anastomosis anchor 236 shown relative to the anastomosis structure 216 described with respect to FIG. 28. As shown, the first annular section 218 is positioned adjacent a stomach wall 238 and the second annular section 220 is positioned adjacent a small intestine wall 240. It will be appreciated that while the anastomosis anchor 236 is shown proximate a braided anastomosis structure 216 such as the Axios® stent, the anastomosis anchor 236 will perform equally well with a different luminal insert or without a stent within the anastomosis 26. For example, the anastomosis structure 216 could instead simply be a pair of magnetic rings, one proximate the stomach wall 238 and one proximate the small intestine wall 240. In some cases, the anastomosis 16 may simply be a surgically (or endoscopically) created structure that is held in place with sutures. The anastomosis 16 may be created in a way that does not require additional structure, like the anastomosis structure 216, to retain patency of the anastomosis 16.

The anastomosis anchor 236 is a self-expanding braided structure including a first expanded diameter portion 242 that is adapted to be secured above the first annular section 218 of the anastomosis structure 216. The anastomosis anchor 236 includes a second expanded diameter portion 244 that is adapted to be secured below the second annular section 220 of the anastomosis structure 216. The anastomosis anchor 236 also includes an intervening portion 246 that extends from the first expanded diameter portion 242 to the second expanded diameter portion 244 and that is adapted to fit within the intermediate portion 222 of the anastomosis structure 216. Terms such as above or below merely refer to the illustrated orientation. The anastomosis structure 236 could be deployed in any orientation, including an orientation that is largely upside down from what is shown in FIG. 29, for example. Moreover, the first expanded diameter portion 242 and the second expanded diameter portion 244 may be considered as being adapted to interact with whatever anastomosis structure is used.

In some cases, the first expanded diameter portion 242 may be designed to be larger than the first annular section 218 of the anastomosis structure 216. The first expanded diameter portion 242 may be large enough to directly engage the stomach wall 238, particularly when a force is applied to the anastomosis anchor 236 via a tether 248. The anastomosis anchor 236 may be formed of materials that are resistant to the gastric environment. The anastomosis anchor 236 may be formed of a shape memory polymer or a shape memory metal. In some cases, the anastomosis anchor 236 may include a covering such as silicone. In some cases, the anastomosis anchor 236 may include hooks or tines that promote anchoring to the stomach wall 238.

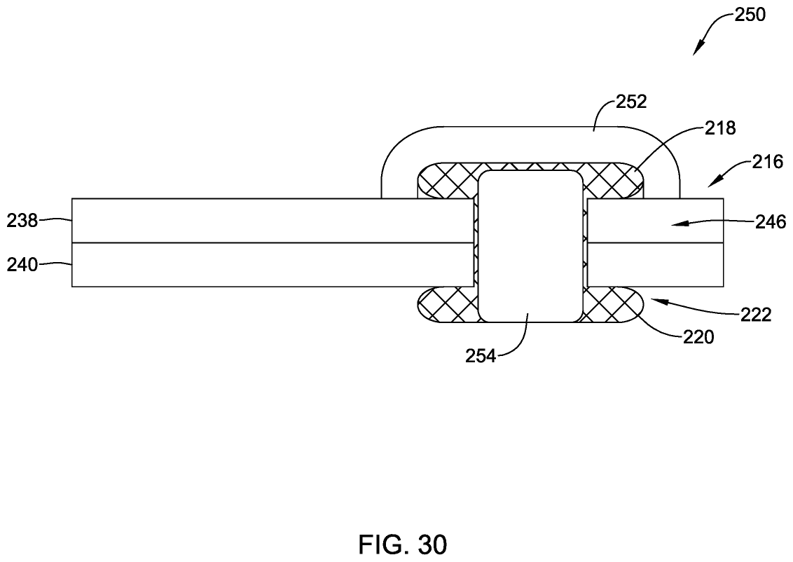

FIG. 30 is a schematic view of an illustrative anastomosis anchor 250 shown relative to the anastomosis structure 216 described with respect to FIG. 28. The anastomosis anchor 250 includes an anchor feature 252 that is adapted to be secured relative to the first annular section 218 of the anastomosis structure 216. The anastomosis anchor 250 also includes a through portion 254 that is coupled with the anchor feature 252 and that is adapted to fit through the intervening portion 222 of the anastomosis structure 216. In some cases, the anchor feature 252 may have one of several different heights, to be able to clear a variety of anastomosis structures. In some cases, the anchor feature 252 may have an annular outer profile. In some cases, the anchor feature 252 may have one, two, three, four or more feet or pads that extend radially outwardly from the anchor feature 252 in order to engage the stomach wall 238.

In some cases, the anastomosis anchor 250 is adapted to form a frictional fit with the first annular section 218 of the anastomosis structure 216. In some cases, the anastomosis anchor 250 includes hooks or tines that are adapted to engage the stomach wall 238. In some cases, the anastomosis anchor 250 includes hooks or tines, or other structure, that are adapted to engage the stomach wall 238 and the through portion 254 includes hooks or tines, or other structure, that is adapted to engage the jejunum wall 240. In some cases, the through portion 254 may include hooks or tines that are adapted to engage the intervening portion 222 of the anastomosis structure 216.

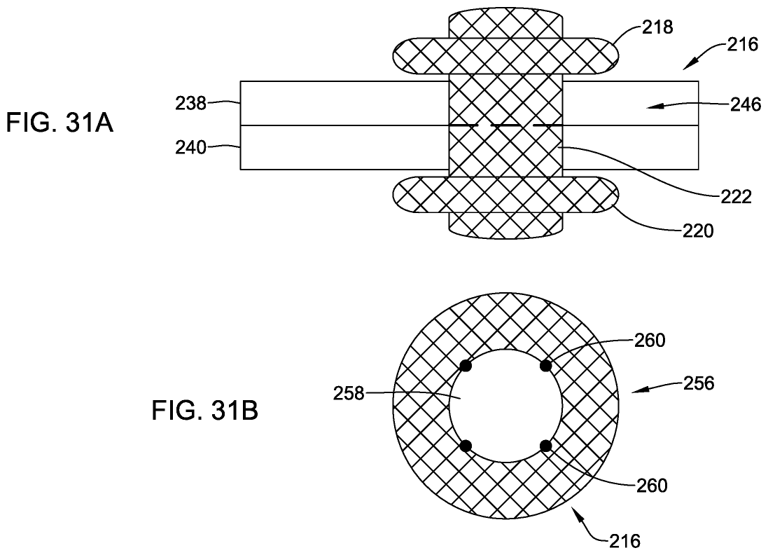

FIGS. 31A and 31B are side and top views, respectively, of an illustrative anastomosis anchor 256 shown disposed within the anastomosis structure 216. As best seen in FIG. 31B, the anastomosis anchor 256 is adapted to fit within the intervening portion 222 of the anastomosis structure 216. In some cases, the anastomosis anchor 256 includes a cylindrical body 258 that optionally includes several axially extending members 260. In some cases, the cylindrical body 258 includes one or more rings that engage with the sides of the anastomosis structure 216. The one or more rings may be telescoping, for example, in order to exert an outward force to help keep the anastomosis structure 216 from migrating. In some cases, a tether may be attached to the anastomosis anchor 256. In some cases, the tether may instead or additionally be attached to the anastomosis structure 216.

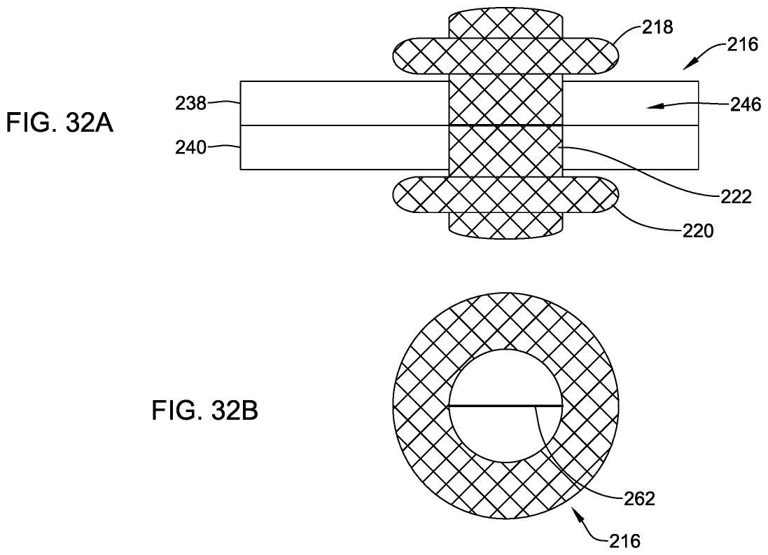

FIGS. 32A and 32B are side and top views, respectively, of an illustrative anastomosis anchor 262 shown disposed within the anastomosis structure 216. As best seen in FIG. 32B, the anastomosis anchor 262 is adapted to fit within the intervening portion 222 of the anastomosis structure 216. In some cases, the anastomosis anchor 262 is a central insert, and may be any four-sided or more than four-sides shape. Examples include but are not limited to cross-sectional profiles defining squares, rectangles and other polygons. The anastomosis anchor 262 may have a rounded shape. The anastomosis anchor 262 may be solid, or may have cutouts to allow chyme to flow through. The anastomosis anchor 262 may have arms or leaves that extend outwardly to help engage the anastomosis structure 216. In some cases, a tether may be attached to the anastomosis anchor 262. In some cases, the tether may instead or additionally be attached to the anastomosis structure 216.

FIG. 33 through FIG. 53D provide examples of illustrative tethers that may be used as the tether 40 as part of the gastric bypass device 34. FIG. 33 is a schematic view of an illustrative gastric bypass device 270. The gastric bypass device 270 includes an occlusion device 272 and an anastomosis anchor 274. A tether 276 extends between the occlusion device 272 and the anastomosis anchor 274. The tether 276 includes a spring 278 that is adapted to provide an increasing return spring in response to elongation of the spring 278 as gastric motion causes movement of the occlusion device 272 and/or the anastomosis anchor 274. In some cases, with the occlusion device 272 and the anastomosis anchor 274 appropriately positioned, the spring 278 is under a small tension. The spring 278 may be formed of any suitable polymeric or metallic material. In some cases, the spring 278 may be formed of Nitinol or stainless steel, for example.

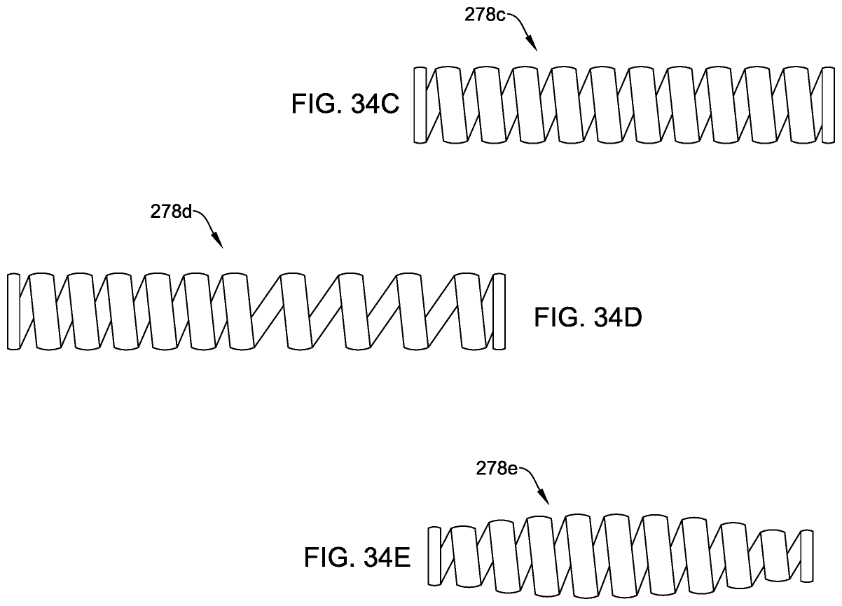

The spring 278 may take a number of forms. FIG. 34A shows a spring 278a having a varying diameter, with a minimum diameter at a midpoint and larger diameters at either end. FIG. 34B shows a spring 278b having a tapering diameter, from a maximum diameter at one end to a minimum diameter at another end. FIG. 34C shows a spring 278c having a uniform diameter and pitch from one end to another end. FIG. 34D shows a spring 278d having a constant outer diameter, but a varying pitch. FIG. 34E shows a spring 278e having a tapering diameter, from a maximum diameter in the middle to a minimum diameter at either end. These are just examples. The springs 280 may be formed of any suitable polymeric or metallic material. In some cases, the springs 280 may be formed of Nitinol or stainless steel, for example.

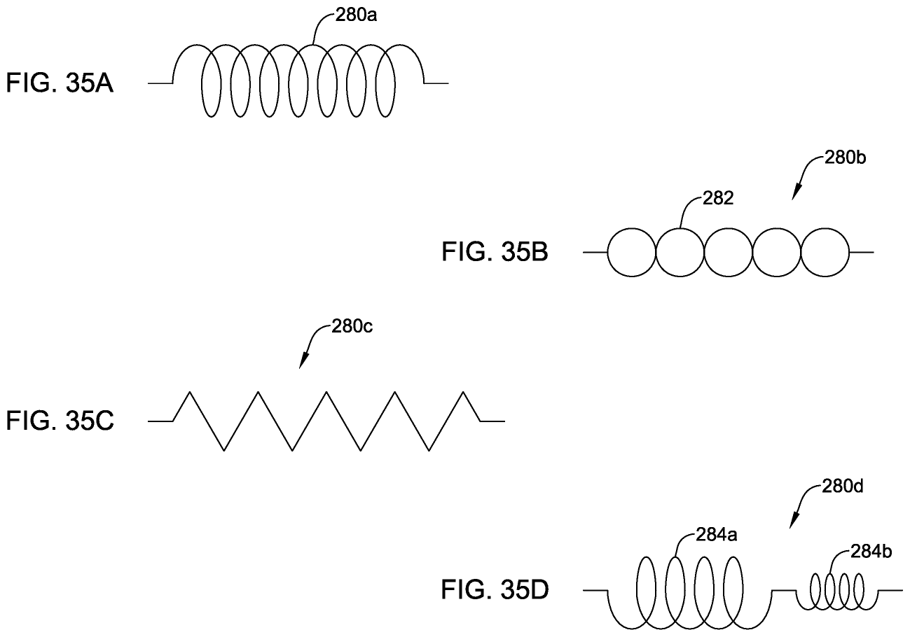

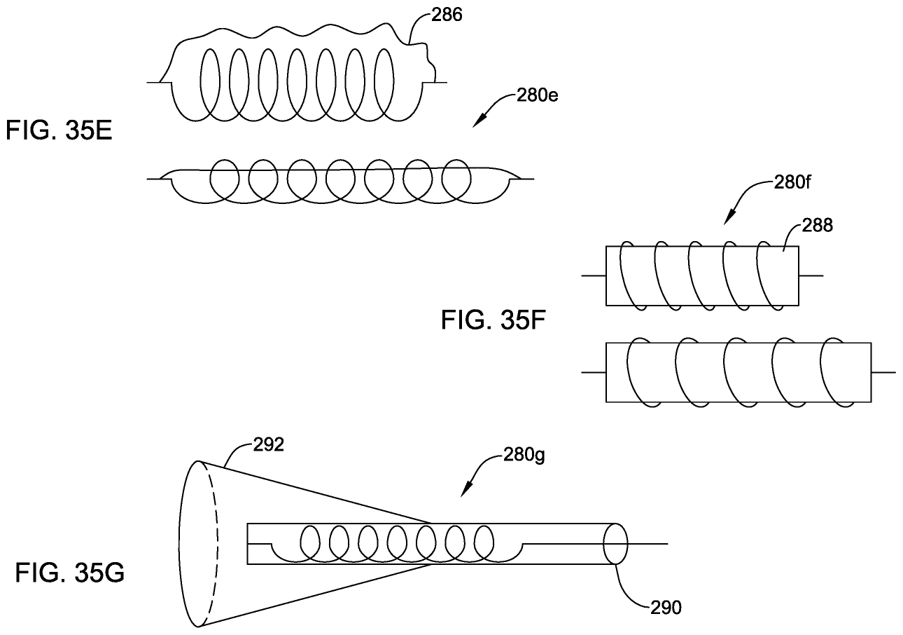

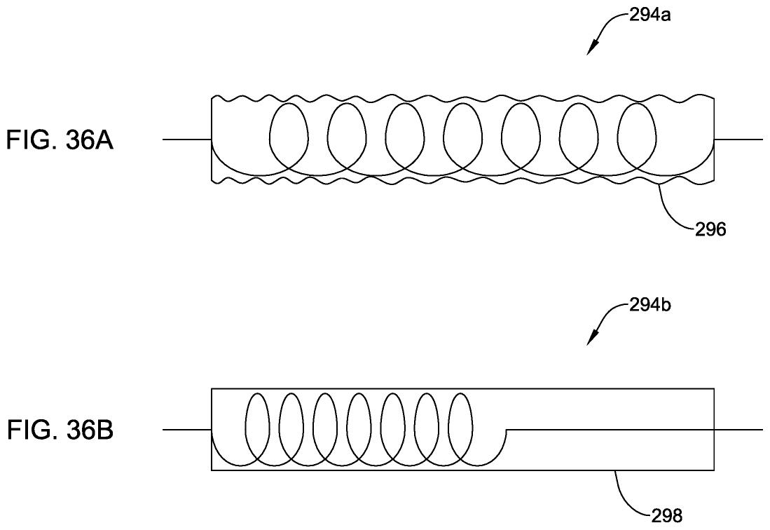

FIG. 35A through 35G show additional possible designs for the spring 278. In FIG. 35A, a spring 280a includes a single, coiled wire. In FIG. 35B, a spring 280b includes joined rings or hoops 282. In FIG. 35C, a spring 280c includes a zig-zag design. In Figure a spring 280d includes a first spring 284a having a first spring constant and a second spring 284b having a second spring constant. The first spring 284a and the second spring 284b may also have differences in other properties such as length and diameter, for example. In Figure a spring 280e may include a string 286 that extends from one end of the spring 280e to the other end of the spring 280e to provide a limit on how far the spring 280e is able to elongate. In FIG. 35F, a spring 280f may be tightly fitted over an inner tube 288 to prevent interactions with food and chyme. In FIG. 35G, a spring 280g may include a stiff tube 290 that extends into an occluder cone 292 in order to get more spring length in a relatively short device. The springs 280 may be formed of any suitable polymeric or metallic material. In some cases, the spring 280 may be formed of Nitinol or stainless steel, for example.

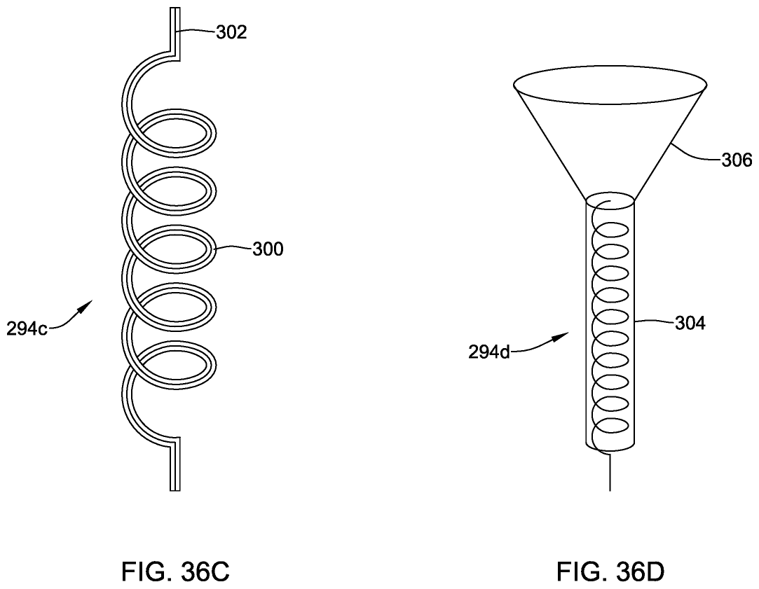

In some cases, the spring or springs may include a covering or coating. A covering or coating may reduce friction or other interactions with tissue within the gastric system. A covering or coating may reduce spring interactions with chyme and food, thereby avoiding possibly clogging. A covering or coating may serve as a barrier to the harsh gastric environment. A covering or coating may reduce damage or inflammation at the bile duct and/or at the papilla. In some cases, a covering or coating may be ePTFE, PTFE or other polymers. FIG. 36A shows a spring 294a having a covering 296 that encapsulates the spring 294a and expands and contracts with the spring 294a. FIG. 36b shows a spring 294b having a covering 298 that allows the spring 294b to move independently of the covering 298. FIG. 36C shows a spring 294c with a covering 300 that is conformal to the filar 302 forming the spring 294c. FIG. 36D shows a spring 294d having a covering 304 that is contiguous with a material 306 forming at least part of the occluding device. The springs 294 may be formed of any suitable polymeric or metallic material. In some cases, the spring 294 may be formed of Nitinol or stainless steel, for example.

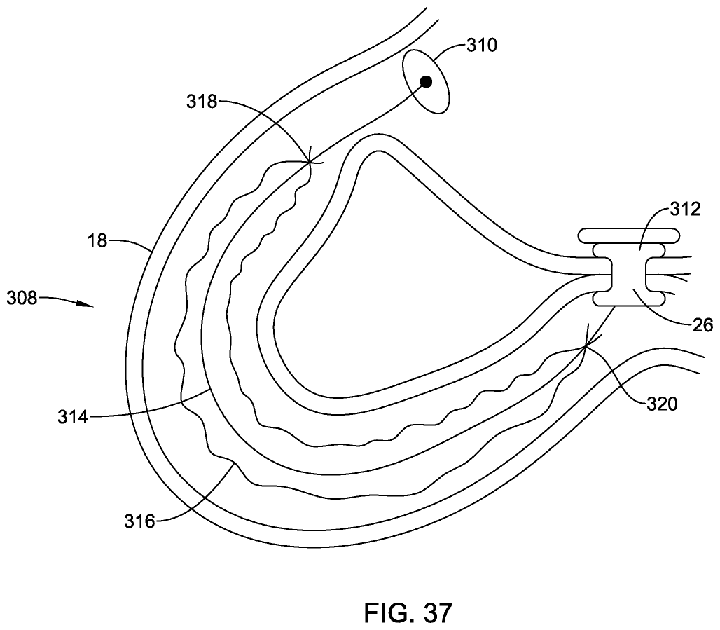

FIG. 37 shows a tether 308 in position within the duodenum 18, extending between an occluding device 310 and an anastomosis anchor 312 shown disposed within the anastomosis 26. The tether 308 includes an inner tether 314 disposed within an anti-corrosive and impermeable sleeve 316 that envelops the inner tether 314. The sleeve 316 protects the inner tether 314 from the gastric environment, while the inner tether 314 provides a tensile force. The sleeve 316 is sealed to the inner tether 314 at a first sealing point 318 and at a second sealing point 320. In between the first sealing point 318 and the second sealing point 320, the inner tether 314 is protected from the gastric environment by the sleeve 316. While the first sealing point 318 is shown distal of the occluding device 310 and the second sealing point 318 is shown proximal of the anastomosis anchor 312, in some cases the sleeve 316 may extend the entire length of the inner tether 314.

In some cases, having the inner tether 314 within the sleeve 316 provides benefits in being able to decouple mechanical and chemical performance. The inner tether 314 may be made from a particular material selected for its mechanical performance without having to worry about whether that material can withstand the harsh gastric environment. This means that any material may be used for forming the inner tether 314.

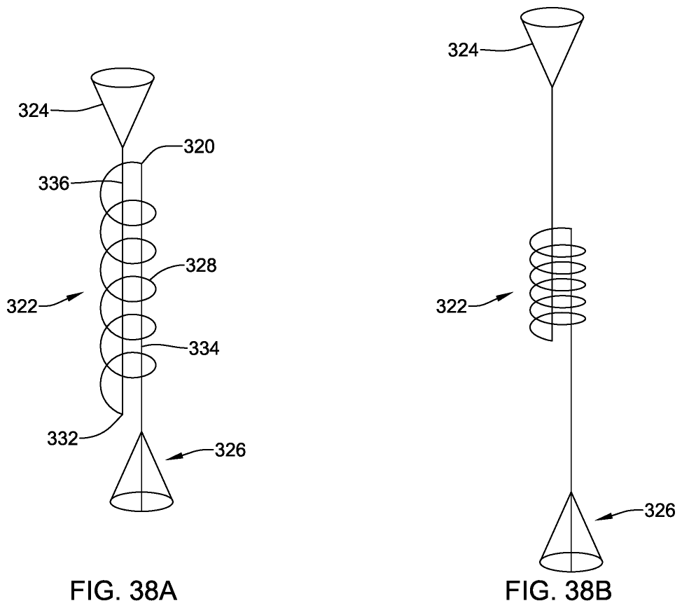

FIGS. 38A and 38B show a tether 322 that extends between an occlusion device 324 and an anastomosis anchor 326. The tether 322 includes a spring 328 having a first end 330 closest to the occlusion device 324 and a second end 332 closest to the anastomosis anchor 326. A first attachment member 334 extends from the first end 330 of the spring 328 to the anastomosis anchor 326. A second attachment member 336 extends from the second end 332 of the spring 328 to the occlusion device 324. As a result, distal movement of the occlusion device 324 and/or proximal movement of the anastomosis anchor 326 will cause compression of the spring 328, as shown in FIG. 38B. This provides a hard-stop to how far apart the occlusion device 324 and the anastomosis anchor 326 can move, because the spring 328 can only be compressed so far. The spring 328 may be formed of any suitable polymeric or metallic material, including NiTi or stainless steel. The spring 328 may vary along its length in diameter. The spring 328 may be a single spring, or the spring 328 may include two or more distinct spring segments.

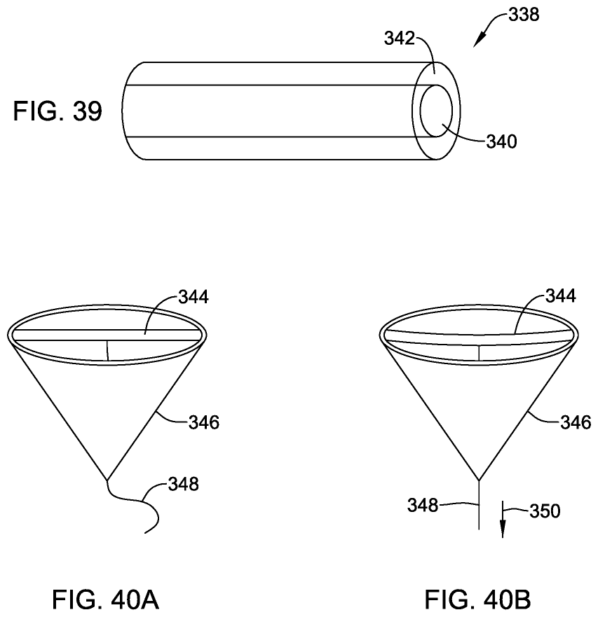

FIG. 39 shows a tether 338 that includes an elastic polymer tube 340 and a covering 342 that covers the elastic polymer tube 340. Elongation of the tether 338 as a result of gastric motion will cause the elastic polymer tube 340 to exert a tensile force as the elastic polymer tube 340 attempts to return to its native or biased configuration. The durometer of the polymer used to form the elastic polymer tube 340 may be varied to adjust its memory force. A variety of polymers may be used for the elastic polymer tube 340. As an example, the elastic polymer tube 340 may be formed of latex. The elastic polymer tube 340 may be a single polymer tube. In some cases, the elastic polymer tube 340 may be a plurality of elastic polymer strands. The covering 342 may help serve as a barrier to the corrosive gastric environment. The covering 342 may reduce interactions with chyme and food particles, and may reduce friction or other interactions with tissue within the gastric system. The covering 342 may reduce damage or inflammation at the bile duct and/or the papilla. In some cases, the covering 342 may be formed of silicone. In some cases, the covering 342 may be PTFE or ePTFE.

In some cases, a spring such as a leaf spring may not be part of the tether itself, but may be attached to either the occlusion device or the anastomosis anchor, with the tether extending from the leaf spring. As a result, tension within the tether will cause the leaf spring to move from its native, biased configuration. FIGS. 40A and 40B show a leaf spring 344 that is secured relative to an occlusion device 346. The leaf spring 344 may be flat, concave or convex. The leaf spring 344 may be flat or round beam, or may have multiple stacked beams. The leaf spring 344 may be formed of any suitable metallic or polymeric material.

A tether 348 extends from the leaf spring 344, through the occlusion device 346 and extends distally therefrom. While the leaf spring 344 is shown attached to the occlusion device 346, a similar result may be achieved by instead securing the leaf spring 344 to an anastomosis anchor. In FIG. 40A, the tether 348 is not under any tension, and the leaf spring 344 remains in a linear configuration, representing a native, biased configuration of the leaf spring 344. In FIG. 40B, the tether 348 is under tension, as indicated by an arrow 350. As can be seen, the leaf spring 344 has bowed, moving out of its native, biased configuration. As a result, the leaf spring 344 will attempt to return to its native configuration, thereby resisting movement of the anastomosis anchor.

In some cases, instead of a leaf spring 344, a spiral torsion spring can be used at an end of a tether. A spiral torsion spring may be secured between the occlusion device 346 and the tether 348. In some cases, a spiral torsion spring may instead be secured between an anastomosis anchor and the tether 348. As the tether 348 provides a tensile force to the spiral torsion spring as a result of gastric movement causing movement of the occlusion device 346 and/or the anastomosis anchor, the spiral torsion spring will move out of its native, biased configuration. As a result, the spiral torsion spring will exert a force on the tether 348 as the spiral torsion spring attempts to regain its native, biased configuration.

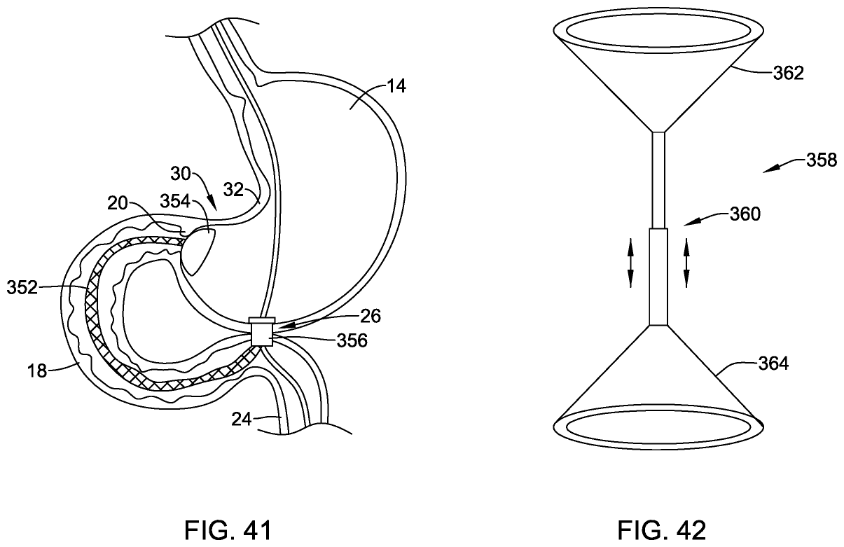

FIG. 41 is a schematic view of an illustrative tether 352 extending between an occlusion device 354 and an anastomosis anchor 356. In some cases, the tether 352 may be formed as a braided stent. The tether 352 may act as a spring, providing a return force in response to elongation of the tether 352 as a result of gastric motion. The tether 352 may be formed of any suitable metallic or polymeric material. The materials, dimensions, pitch, etc. of the tether 352 may be varied in order to provide desired return force behavior. As an example, the tether 352 may be designed to provide a linear increase in return force with device elongation. The tether 352 may be designed to provide an increase in return force with device elongation, for example.

In some cases, the tether 352 may include a coating or covering that helps to protect the tether 353 from the gastric environment. If included, the coating or covering may reduce interactions with chyme and food particles, can reduce friction and interactions with gastric tissue, and can reduce damage or inflammation at the bile duct and/or the papilla. If included, the coating or covering may be any suitable material such as but not limited to PTFE and ePTFE.

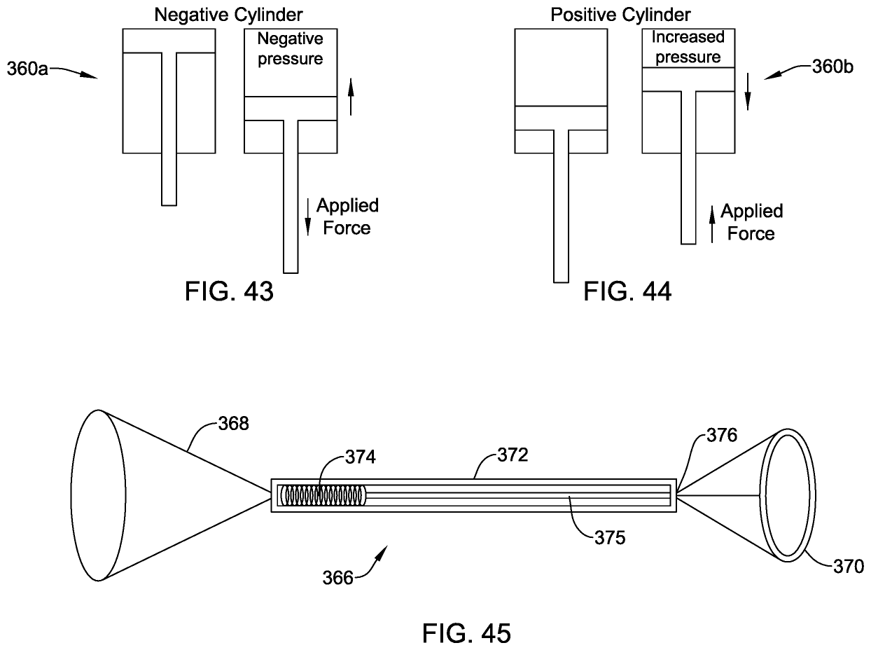

FIG. 42 is a schematic view of an illustrative tether 358 that is formed as or otherwise includes a pneumatic cylinder 360. The tether 358 extends between an occlusion device 362 and an anastomosis anchor 364. The pneumatic cylinder 360 provides a return force in response to a tensile force being applied to the tether 358. In some cases, the pneumatic cylinder 360 may be a positive cylinder or a negative cylinder. The pneumatic cylinder 360 may be rigid or flexible, and may be located anywhere along the length of the tether 358, from near the occlusion device 362, near the anastomosis anchor 364 or anywhere in between. The pneumatic cylinder 360 may be metallic or polymeric, and may be filled with a liquid or gas working fluid. FIG. 43 shows an example of a negative cylinder 360a while FIG. 44 shows an example of a positive cylinder 360b.

FIG. 45 is a schematic view of an illustrative tether 366 extending between an occlusion device 368 and an anastomosis anchor 370. The tether 366 includes a protective sleeve 372, a spring 374 and a sliding joint 376 that allows the spring 374 to elongate within, but independently from the protective sleeve 372. In some cases, the tether 366 includes a PTFE tube 375. In some cases, the PTFE tube 375 allows the tether 366 to smoothly elongate, moving in and out of the sliding joint 376. In some cases, there may be a Nitinol wire inside the PTFE tube 375 to prevent kinking. There may also be a suture inside the PTFE tube 375 that is attached to the spring 374 and to the anastomosis anchor 370, and holds the spring 374 in place. This suture may be formed of UHMWPE (ultra high molecular weight polyethylene). While the spring 374 is shown proximate the occlusion device 368, the spring 374 may alternative be located proximate the anastomosis anchor 370. The sliding joint 376 is low friction, and thus allows travel at low applied forces, while being tightly fit to minimize the likelihood of chyme entering the protective sleeve 372. In some cases, the sliding joint 376 may be a small tube within a larger tube. The sliding joint 376 may be a small tube pulled through a soft membrane. The sliding joint 370 may be one or multiple threads within a tube. The threads may be polymeric or metallic. The threads may be single-stranded, multiple-stranded, or braided together. The spring may be elastic polymer or a metal such as Nitinol or spring steel. The protective sleeve 372 may be formed of a flexible, durable and corrosion-resistant polymer such as ePTFE.

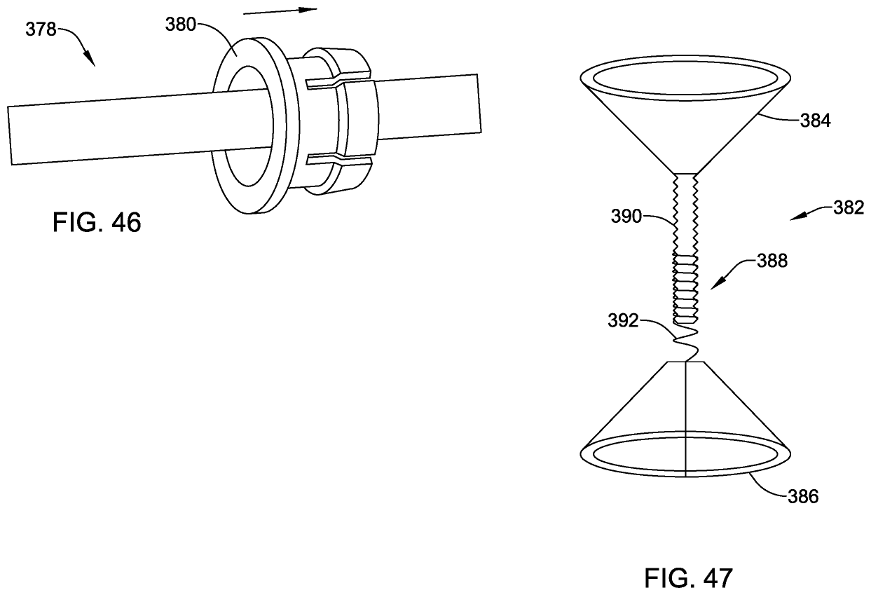

FIG. 46 is a schematic view of an illustrative tether 378 that includes a collet 380. In some cases, the collet 380 is a one-way collet, adapted to allow the tether 378 to be pulled through in a first direction while preventing travel of the tether 378 in the opposing direction. In some cases, the tether 378 may be pulled relative to the collet 380 to shorten the tether 378. In some cases, the collet 380 may include teeth or barbs that allow the tether 378 to travel in one direction but not the opposite direction. The collet 380 may utilize frictional forces to control travel. In some cases, the collet 380 may mate with interlocking features on the tether 378 to control travel. The collet 380 may be located near the occlusion device, near the anastomosis anchor or anywhere in between. The collet 380 enables in-vivo tether length adjustment by the physician in order to adjust for a specific patient's anatomy. In some cases, the collet 380 may be adjusted on the bench-top, prior to implantation, in order to adjust the effective length of the tether 378.

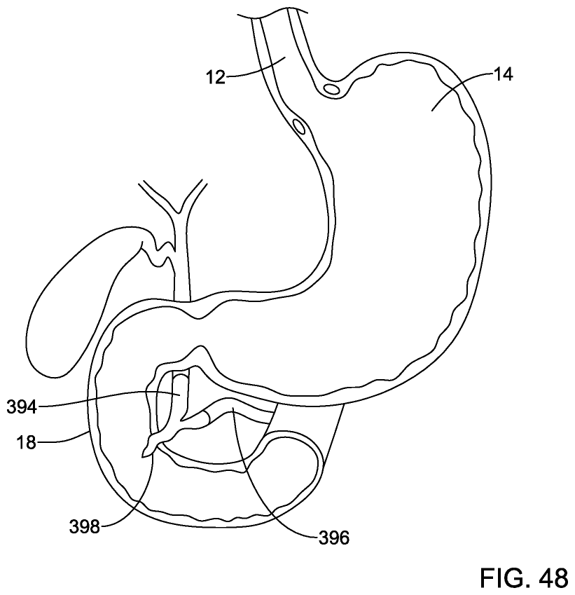

FIG. 47 is a schematic view of an illustrative tether 382 extending between an occlusion device 384 and an anastomosis anchor 386. The tether 382 includes a threaded joint 388 between a threaded member 390 and a spring 392 that threadedly engages the threaded member 390. In some cases, the threaded joint 388 may be adjusted on the bench-top, prior to implantation, in order to adjust the effective length of the tether 382. In some cases, the threaded joint 388 may be adjusted in-vivo using endoscopic tools. The tether 382 may be formed of any suitable materials. The threaded joint 388 may be located near the occlusion device 384, near the anastomosis anchor 386, or anywhere in between.

FIG. 48 is a schematic view of a portion of the gastrointestinal system that indicates the relative location of a patient's bile duct 394, the patient's pancreatic duct 396 and the patient's Papilla of Vater 398. The Papilla of Vater 398 is where the bile duct 394 and the pancreatic duct 396 are fluidly coupled with the duodenum 18. The Papilla of Vater 398 is located on an inside curve of the duodenum 18, and in some instances may protrude part way into the interior of the duodenum 18. A possible issue with placing a tether in the duodenum 18 is that the tether may irritate the Papilla of Vater 398, which can cause inflammation and in turn a variety of possible complications. In some cases, there is a desire to provide tethers that avoid irritating the Papilla of Vater 398.

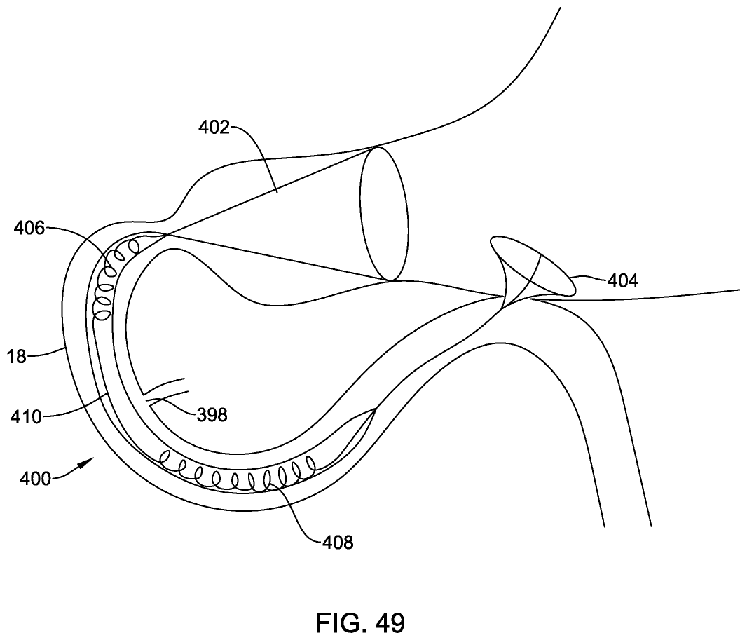

FIG. 49 is a schematic view of an illustrative tether 400 extending through the duodenum 18 between an occlusion device 402 and an anastomosis anchor 404. As shown, the tether 400 includes a first spring segment 406 that is proximal of the Papilla of Vater 398 and a second spring segment 408 that is distal of the Papilla of Vater 398. The tether 400 includes a member 410 that extends between the first spring segment 406 and the second spring segment 408, and passes over the Papilla of Vater 398. Because the first spring segment 406 and the second spring segment 408 have a larger diameter (although not necessarily the same as each other) relative to the member 410, the member 410 is held off the interior surface of the duodenum 18 and thus away from the Papilla of Vater 398.

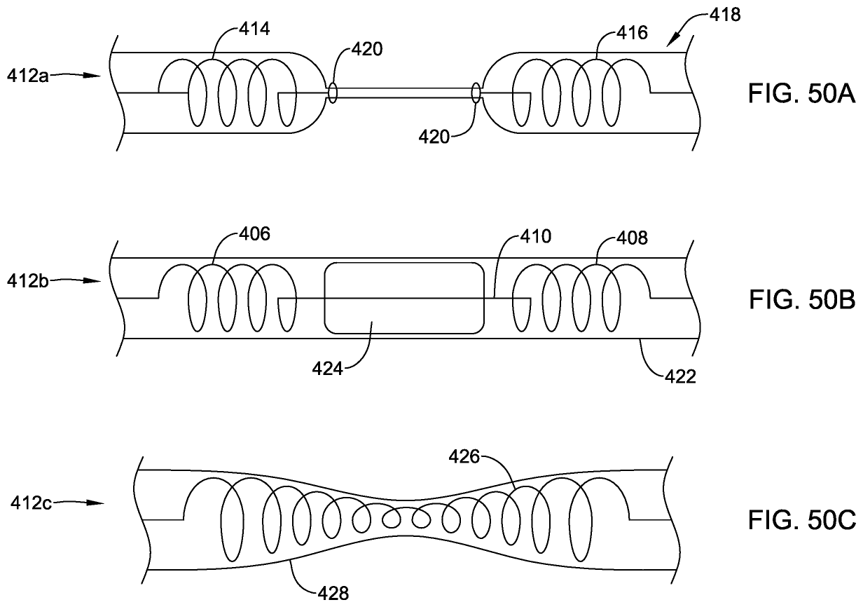

FIGS. 50A, 50B and 50C provide additional examples. In FIG. 50A, a tether 412a includes a first spring segment 414 and a second spring segment 416, but does not include any metallic structure therebetween. Instead, the tether 412a includes an atraumatic covering 418 that encapsulates the first spring segment 414 and the second spring segment 416. The atraumatic covering 418 is narrowed between the first spring segment 414 and the second spring segment 416 via a pair of sutures 420 that secure the atraumatic covering 418 to an end of the first spring segment 414 and to an end of the second spring segment 416. Thus, nothing metallic will come near the Papilla of Vater 398.

FIG. 50B shows a tether 412b that is similar to the tether 400, but includes an atraumatic covering 422 that encapsulates the first spring segment 406, the second spring segment 408 and the member 410 extending therebetween. In some cases, as shown, a soft pillow 424 is disposed between the first spring segment 406 and the second spring segment 408 and is held in place by the atraumatic covering 422. Thus, nothing metallic will come near the Papilla of Vater 398. FIG. 50C shows a tether 412c that includes a spring 426 disposed within an atraumatic covering 428. The spring 426 has a varying diameter, with a maximum diameter at either end of the spring 426 and tapering to minimal diameter near a midpoint of the spring 426.

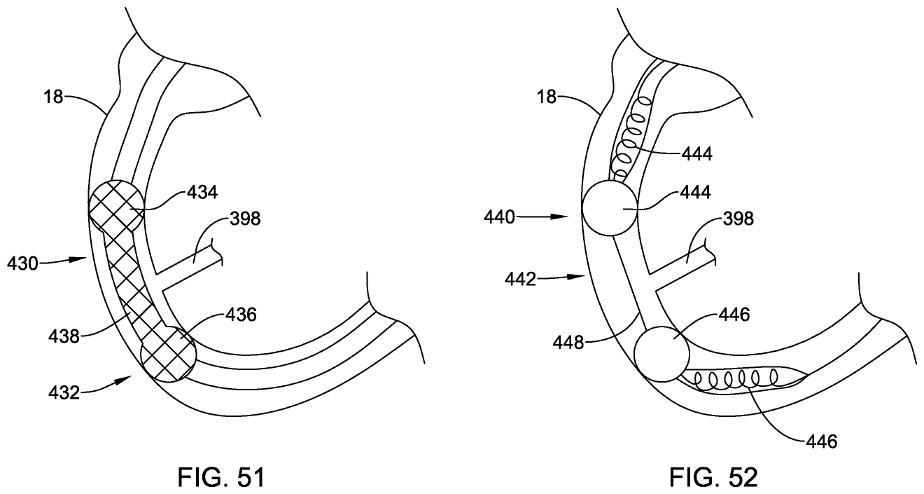

FIG. 51 shows a tether 430 that includes a physical standoff 432 disposed over the tether 430, with the tether 430 extending through the physical standoff 432. In some cases, the physical standoff 432 is able to slide relative to the tether 430. In some cases, the physical standoff 432 is a braided structure formed of a metal such as Nitinol. In some cases, the physical standoff 432 may instead be inflatable, such as an inflatable balloon. As shown, the physical standoff 432 includes a first bulb region 434 and a second bulb region 436, with a narrowed diameter portion 438 extending between the first bulb region 434 and the second bulb region 436. In some cases, the physical standoff 432 may further include additional bulb regions.

FIG. 52 shows a tether 440 that includes a physical standoff 442 that forms a part of the tether 440. The tether 440 includes a first spring segment 444 and a second spring segment 446, with the physical standoff 442 disposed between the first spring segment 444 and the second spring segment 446. The physical standoff 442 may be welded or sewn or bonded to each of the first spring segment 444 and the second spring segment 446, for example. The physical standoff 442 may include a first bulb region 444 and a second bulb region 446, with a stiff member 448 extending between the first bulb region 444 and the second bulb region 446.

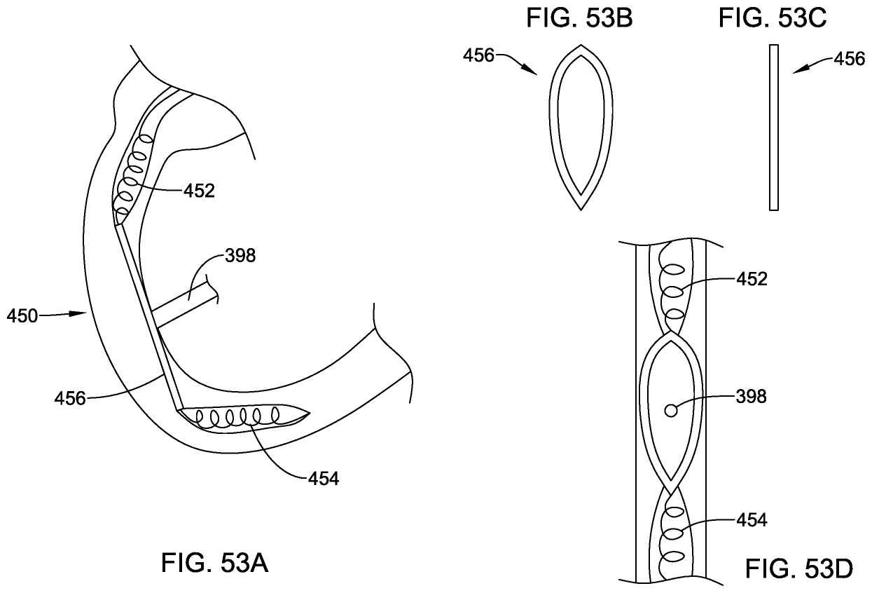

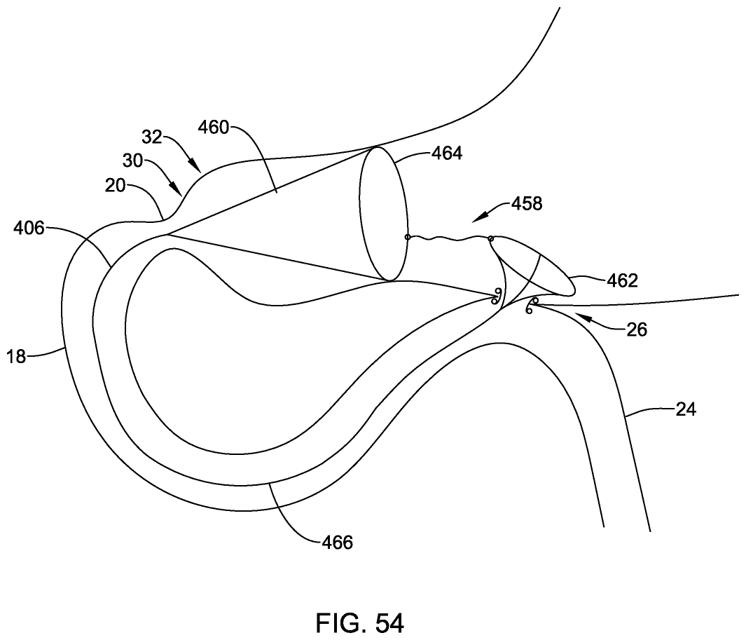

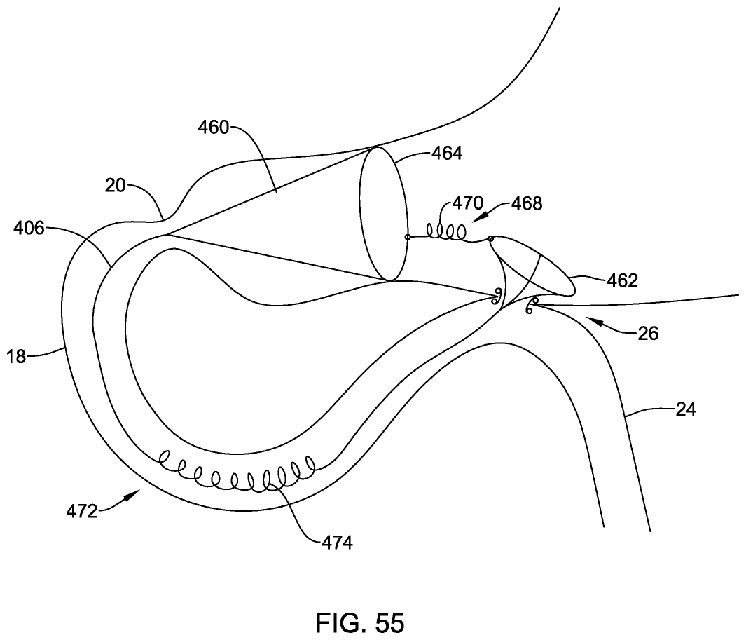

FIG. 53A is a schematic view of an illustrative tether 450. The illustrative tether 450 includes a first spring segment 452 and a second spring segment 454. The tether 450 includes a bow segment 456 that extends between the first spring segment 452 and the second spring segment 454. FIG. 53B shows a first view of the bow segment 456 while FIG. 53C shows a second view of the bow segment 456. Tension on the tether 450 will rotate the bow segment 456 perpendicular to the Papilla of Vater 398, as shown in FIG. 53D.

In some cases, a dynamic leash may be used as part of a gastric bypass device. FIG. 54 through FIG. 59 provide examples of illustrative dynamic leashes that may be used as the dynamic leash 42 as part of the gastric bypass device 34.

FIG. 54 is a schematic view of an illustrative dynamic leash 458 that is shown within the anatomy. The dynamic leash 458 extends between an occlusion device 460 that is disposed proximate the pyloric sphincter 20 and an anastomosis anchor 462 that is disposed proximate the anastomosis 26. As shown, the dynamic leash 458 is secured to an annular ring 464 forming a part of the occlusion device 460. A tether 466 also extends between the occlusion device 460 and the anastomosis anchor 462 through the duodenum 18. It will be appreciated that the tether 466 and the dynamic leash 458 may work together to help hold the occlusion device 460 in place, regardless of how gastric motion attempts to dislodge the occlusion device 460. If gastric motion attempts to move the occlusion device 460 proximally, into the stomach 14, the tether 466 will provide a resistive force to that motion. If gastric motion attempts to move the occlusion device 460 distally, into the duodenum 18, the dynamic leash 458 will provide a resistive force to that motion.

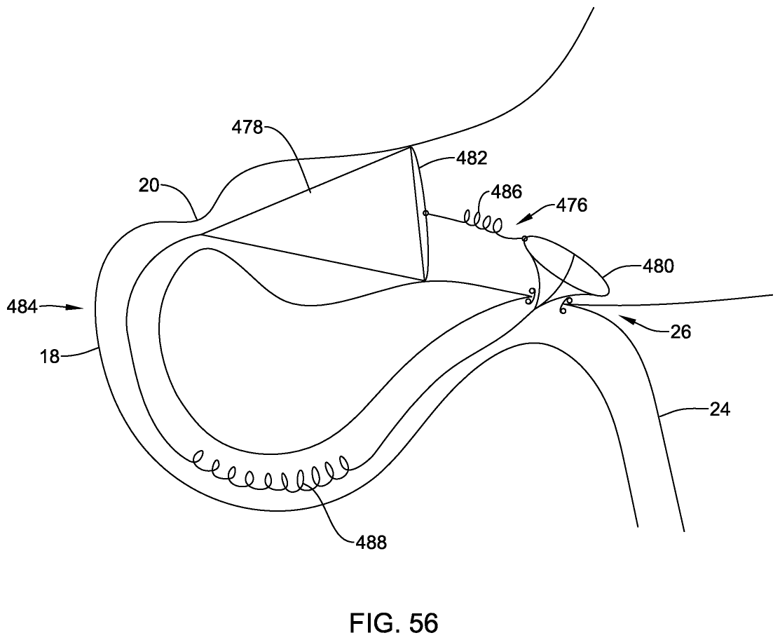

FIG. 55 is a schematic view of an illustrative dynamic leash 468 shown within the anatomy. The dynamic leash 468 includes a spring 470. A tether 472 includes a spring 474. In some cases, the spring 470 has a first spring constant and the spring 474 has a second spring constant. In some cases, the spring 470 and the spring 474 may be selected as a combination, to ensure that the two springs 470 and 474 together provide a dynamic equilibrium force to maintain the desired location of the occlusion device 460.

FIG. 56 is a schematic view of an illustrative dynamic leash 476 that is shown within the anatomy. The dynamic leash 476 extends between an occlusion device 478 that is disposed proximate the pyloric sphincter 20 and an anastomosis anchor 480 that is disposed proximate the anastomosis 26. As shown, the dynamic leash 476 is secured to a cover 482 forming a part of the occlusion device 478. In some cases, a central attachment point such as to the cover 482 results in allowing the occlusion device 478 to lay flat while attaching to the side of the occlusion device 478 may cause the occlusion device 478 to tilt or pivot in place.

A tether 484 also extends between the occlusion device 478 and the anastomosis anchor 480 through the duodenum 18. It will be appreciated that the tether 484 and the dynamic leash 476 may work together to help hold the occlusion device 478 in place, regardless of how gastric motion attempts to dislodge the occlusion device 478. If gastric motion attempts to move the occlusion device 478 proximally, into the stomach 14, the tether 484 will provide a resistive force to that motion. If gastric motion attempts to move the occlusion device 478 distally, into the duodenum 18, the dynamic leash 476 will provide a resistive force to that motion. In some cases, the dynamic leash 476 includes a spring 486. The tether 484 includes a spring 488. In some cases, the spring 486 has a first spring constant and the spring 488 has a second spring constant. In some cases, the spring 486 and the spring 488 may be selected as a combination, to ensure that the two springs 486 and 488 together provide a dynamic equilibrium force to maintain the desired location of the occlusion device 478.

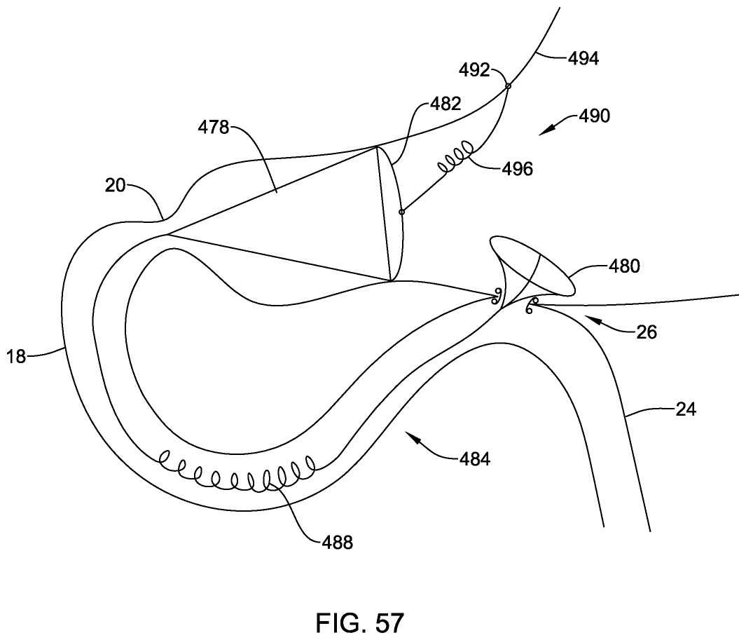

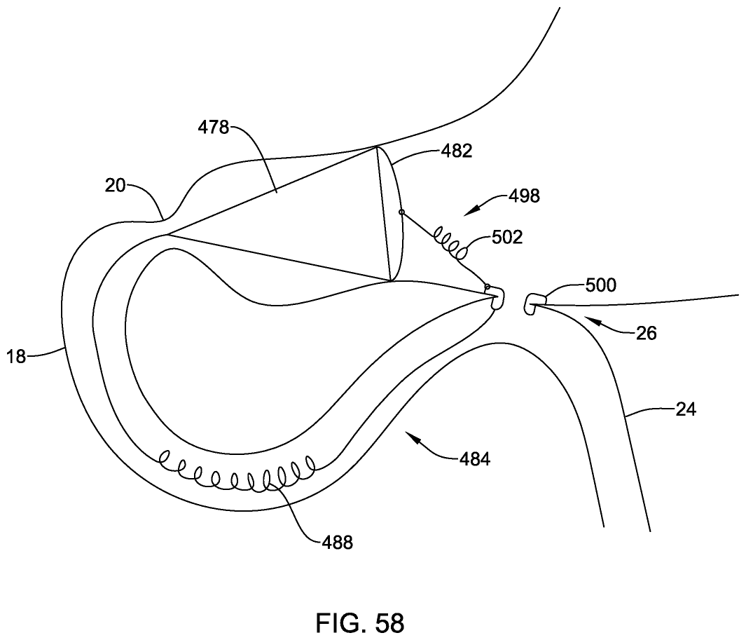

FIG. 57 is a schematic view of an illustrative dynamic leash 490 that is shown within the anatomy. The dynamic leash 490 extends between the occlusion device 478 that is disposed proximate the pyloric sphincter 20 and an attachment point 492 within the stomach wall 494. As shown, the dynamic leash 476 is secured to the cover 482 forming a part of the occlusion device 478. In some cases, a central attachment point such as to the cover 482 results in allowing the occlusion device 478 to lay flat while attaching to the side of the occlusion device 478 may cause the occlusion device 478 to tilt or pivot in place.

A tether 484 extends between the occlusion device 478 and the anastomosis anchor 480 through the duodenum 18. It will be appreciated that the tether 484 and the dynamic leash 490 may work together to help hold the occlusion device 478 in place, regardless of how gastric motion attempts to dislodge the occlusion device 478. If gastric motion attempts to move the occlusion device 478 proximally, into the stomach 14, the tether 484 will provide a resistive force to that motion. If gastric motion attempts to move the occlusion device 478 distally, into the duodenum 18, the dynamic leash 490 will provide a resistive force to that motion. In some cases, the dynamic leash 490 includes a spring 496. In some cases, the spring 496 has a first spring constant and the spring 488 has a second spring constant. In some cases, the spring 496 and the spring 488 may be selected as a combination, to ensure that the two springs 496 and 488 together provide a dynamic equilibrium force to maintain the desired location of the occlusion device 478.

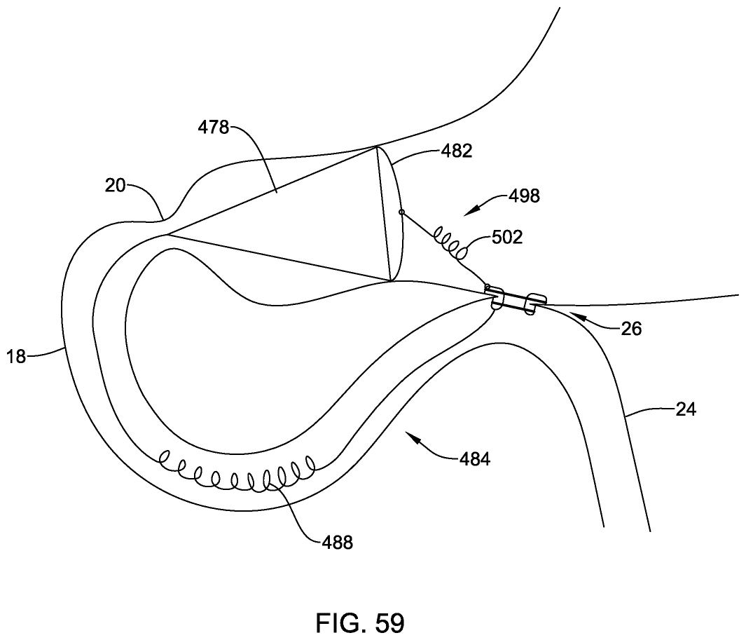

FIG. 58 is a schematic view of an illustrative dynamic leash 498 that is shown within the anatomy. The dynamic leash 498 extends between an occlusion device 478 that is disposed proximate the pyloric sphincter 20 and an antimigration anchor 500 that is disposed proximate the anastomosis 26. An antimigration anchor 500 is an anchor that may be attached to the stomach 14 such that tension can be applied on the anchor in either direction without the antimigration anchor moving. In some cases, the dynamic leash 498 may connect to the antimigration anchor 500 such that tension from the dynamic leash 498 may cause the antimigration anchor 500 to change in diameter and/or shape. As an example, the antimigration anchor 500 may increase in diameter and decrease in length so as to provide additional outward radial force and prevent slippage through the anastomosis 16. As shown, the dynamic leash 498 is secured to a cover 482 forming a part of the occlusion device 478. In some cases, a central attachment point such as to the cover 482 results in allowing the occlusion device 478 to lay flat while attaching to the side of the occlusion device 478 may cause the occlusion device 478 to tilt or pivot in place.

The tether 484 extends between the occlusion device 478 and the antimigration anchor 500 through the duodenum 18. It will be appreciated that the tether 484 and the dynamic leash 498 may work together to help hold the occlusion device 478 in place, regardless of how gastric motion attempts to dislodge the occlusion device 478. If gastric motion attempts to move the occlusion device 478 proximally, into the stomach 14, the tether 484 will provide a resistive force to that motion. If gastric motion attempts to move the occlusion device 478 distally, into the duodenum 18, the dynamic leash 498 will provide a resistive force to that motion. In some cases, the dynamic leash 498 includes a spring 502. In some cases, the spring 502 has a first spring constant and the spring 488 has a second spring constant. In some cases, the spring 502 and the spring 488 may be selected as a combination, to ensure that the two springs 502 and 488 together provide a dynamic equilibrium force to maintain the desired location of the occlusion device 478.

FIG. 59 is a schematic view of the illustrative dynamic leash 498 that is shown within the anatomy. The dynamic leash 498 extends between the occlusion device 478 that is disposed proximate the pyloric sphincter 20 and a pair of magnet rings 506 that is disposed proximate the anastomosis 26. The tether 484 extends between the occlusion device 478 and the magnet rings 506 through the duodenum 18. It will be appreciated that the tether 484 and the dynamic leash 498 may work together to help hold the occlusion device 478 in place, regardless of how gastric motion attempts to dislodge the occlusion device 478. If gastric motion attempts to move the occlusion device 478 proximally, into the stomach 14, the tether 484 will provide a resistive force to that motion. If gastric motion attempts to move the occlusion device 478 distally, into the duodenum 18, the dynamic leash 476 will provide a resistive force to that motion.

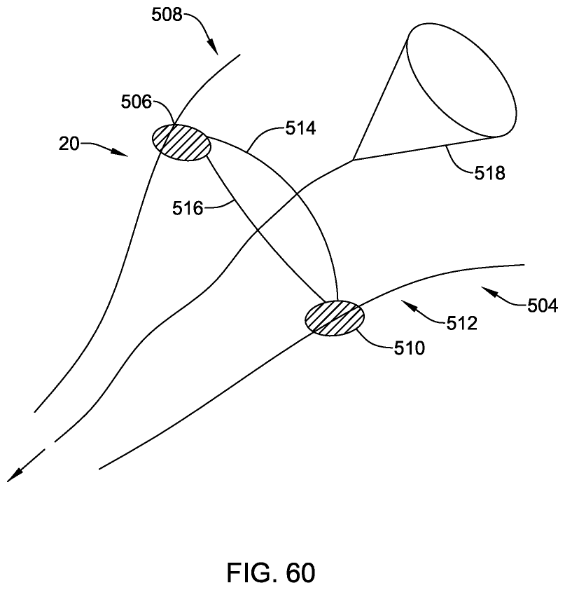

FIG. 60 is a schematic view of a passive engagement apparatus 504 shown proximate the pyloric sphincter 20. The passive engagement apparatus 504 includes a first gastric clip 506 that is securable on a first side 508 of the pyloric sphincter 20, the pylorus 30 or the antrum 32 and a second gastric clip 510 that is securable on a second side 512 of the pyloric sphincter 20, the pylorus 30 or the antrum 32. A first elastic band 514 extends between the first gastric clip 506 and the second gastric clip 510. A second elastic band 516 extends between the first gastric clip 506 and the second gastric clip 510. Together, the first elastic band 514 and the second elastic band 516 help to prevent an occlusion device 518 from moving distally. As the pyloric sphincter 20 expands, the first elastic band 514 and the second elastic band 516 will engage with the occlusion device 518 and prevent distal movement of the occlusion device 518. When the pyloric sphincter 20 is not expanding, or is relaxed, the first elastic band 514 and the second elastic band 516 do not contact the occlusion device 518.

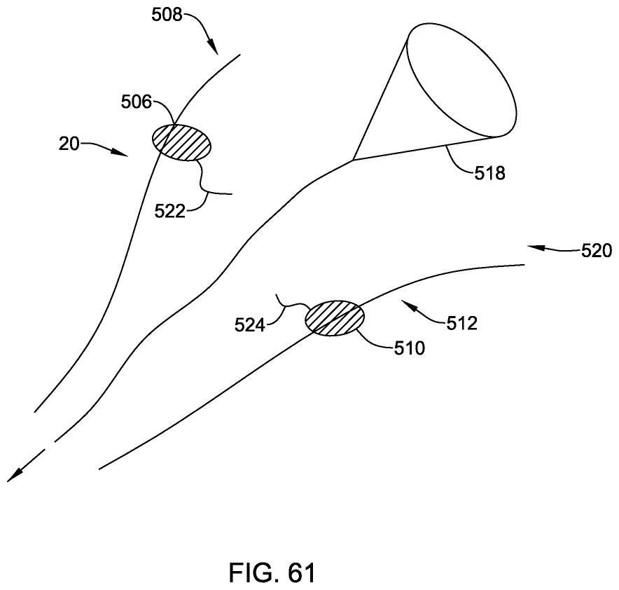

FIG. 61 is a schematic view of a passive engagement apparatus 520 shown proximate the pyloric sphincter 20. The passive engagement apparatus 520 includes the first gastric clip 506 that is securable on the first side 508 of the pyloric sphincter 20, the pylorus 30 or the antrum 32 and the second gastric clip 510 that is securable on the second side 512 of the pyloric sphincter 20, the pylorus 30 or the antrum 32. A first hook or bumper 522 is attached to the first gastric clip 506 and a second hook or bumper 524 is attached to the second gastric clip 510. Together, the first hook or bumper 522 and the second hook or bumper 524 help to prevent the occlusion device 518 from moving distally. As the pyloric sphincter 20 expands and the occlusion device 518 moves distally, the first hook or bumper 522 and the second hook or bumper 524 will engage with the occlusion device 518 and prevent distal movement of the occlusion device 518. When the pyloric sphincter 20 is not expanding, or is relaxed, the first hook or bumper 522 and the second hook or bumper 524 do not contact the occlusion device 518.

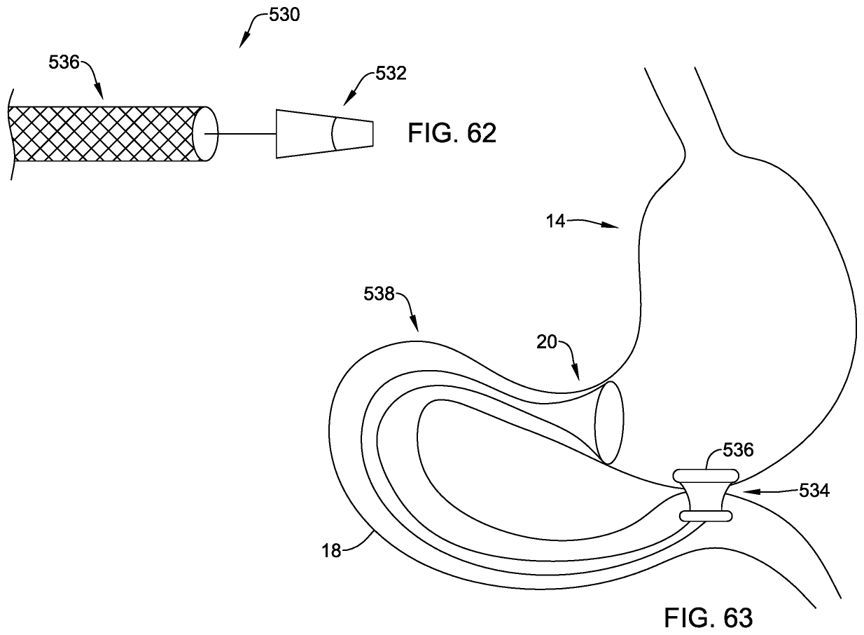

FIG. 62 is a schematic view of an illustrative device 530 that integrates anastomosis creation and gastric bypass system delivery into a single step. The illustrative device 530 includes an electrocautery tip 532 that is used to create an anastomosis 534 as well as a collapsed anastomosis anchor 536 that is deployed after the anastomosis 534 has been created using the electrocautery tip 532. Once the anastomosis 534 has been created, the rest of a gastric bypass device 538 may be delivered through a pull-back process (duodenum 18 to pyloric sphincter 20). A sleeve (not shown) may hold the anastomosis anchor 536 in the collapsed configuration prior to removing the sleeve. The gastric bypass device 538 is shown in FIG. 63, for example.

The materials that can be used for the various components of the medical device systems described herein and the various elements thereof disclosed herein may include those commonly associated with medical devices. In some embodiments, the medical device systems described herein may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as WIP35-N0 and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the medical device systems described herein may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the medical device systems. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device systems described herein.

In some embodiments, a degree of Magnetic Resonance Imaging (MRI) compatibility is imparted into the medical device systems described herein. The medical devices described herein may be made of a material that does not substantially distort the image and create substantial artifacts (e.g., gaps in the image). Certain ferromagnetic materials, for example, may not be suitable because they may create artifacts in an MRI image. In some cases, the medical device systems, or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-NO and the like), nitinol, and the like, and others.

In some embodiments, the medical device systems described herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the medical device systems described herein and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. An implantable medical device system, comprising:
an occlusion device adapted to be secured in place relative to a patient's pylorus;
an anastomosis anchor adapted to be secured in place relative to an anastomosis formed between the patient's stomach wall and the patient's small intestine;
a tether adapted to extend through the patient's duodenum, the tether secured at a first end to the occlusion device and at a second end to the anastomosis anchor, the tether further adapted to provide a tensile force to the occlusion device; and
a dynamic leash adapted to extend through the patient's stomach, the dynamic leash further adapted to provide a tensile force to the occlusion device that is in opposition to that provided by the tether.

2. The implantable medical device system of claim 1, wherein the tether comprises a first spring with a first spring constant and the dynamic leash comprises a second spring with a second spring constant.

3. The implantable medical device system of claim 2, wherein the second spring constant is different from the first spring constant.

4. The implantable medical device system of claim 2, wherein the first spring is adapted to resist gastrointestinal system movements that could otherwise cause the occlusion device to move into the patient's stomach.

5. The implantable medical device system of claim 2, wherein the second spring is adapted to resist gastrointestinal system movements that could otherwise cause the occlusion device to move into the patient's duodenum.

6. The implantable medical device system of claim 2, wherein the first spring is adapted to work together with the second spring to keep the occlusion device in place relative to the patient's pylorus.

7. The implantable medical device system of claim 1, wherein the dynamic leash is adapted to be secured at one end to the occlusion device and at an opposing end to the anastomosis anchor.

8. The implantable medical device system of claim 7, wherein the occlusion device comprises a supporting frame and an impermeable membrane spanning the supporting frame.

9. The implantable medical device system of claim 8, wherein the dynamic leash is adapted to be secured at one end to the supporting frame of the occlusion device.

10. The implantable medical device system of claim 8, wherein the dynamic leash is adapted to be secured at one end to the impermeable membrane of the occlusion device.

11. The implantable medical device system of claim 1, wherein the dynamic leash is adapted to be secured at one end to the occlusion device and at an opposing end to a wall of the patient's stomach.

12. The implantable medical device system of claim 1, wherein the dynamic leash is adapted to be secured at one end to the occlusion device and at an opposing end to an anti-migration anchor.

13. A dynamic leash adapted for use in a gastric bypass system including an occlusion device and a tether secured to the occlusion device, the dynamic leash adapted to extend within a patient's stomach between an occlusion device adapted to be deployed relative to a patient's pylorus and a securement point, the dynamic leash comprising:
an elastic member having a first end adapted to be secured relative to the occlusion device and a second end adapted to be secured to a securement point;
wherein the elastic member dynamically opposes forces placed on the occlusion device by the tether.

14. The dynamic leash of claim 13, wherein the securement point comprises an anastomosis anchor.

15. The dynamic leash of claim 13, wherein the securement point comprises a wall of the patient's stomach.

16. The dynamic leash of claim 13, wherein the elastic member comprises a spring.

17. The dynamic leash of claim 11, wherein the elastic member comprises a pneumatic tube.

18. The dynamic leash of claim 11, wherein the elastic member is adapted to resist gastrointestinal system movements that could otherwise cause the occlusion device to move into the patient's duodenum.

19. A device adapted to create an anastomosis and deliver a gastric bypass system in a single step, the device comprising:
an electrocautery tip adapted to create an anastomosis; and
a device segment adapted to releasably hold an anastomosis anchor in a collapsed configuration;
the device segment adapted to deploy the anastomosis anchor within the anastomosis, the anastomosis anchor expanding to an expanded configuration upon deployment.

20. The device of claim 19, wherein a gastric bypass device may be delivered through a pull-back process.
